(19) United States
(12) Patent Application Publication
(10) Pub. No.: US 2019/0201485 A1
(43) Pub. Date: Jul. 4, 2019

(54) ANTI-FIBROTIC SIALIDASE INHIBITOR COMPOUNDS AND METHODS OF USE

(71) Applicant: The Texas A&M University System, College Station, TX (US)

(72) Inventors: Richard H. Gomer, College Station, TX (US); Darrell Pilling, Cypress, TX (US); Nehemiah Cox, New York, NY (US); Tejas R. Karhadkar, College Station, TX (US)

(21) Appl. No.: 16/293,379

(22) Filed: Mar. 5, 2019

Related U.S. Application Data
(63) Continuation of application No. PCT/US2017/050435, filed on Sep. 7, 2017.
(60) Provisional application No. 62/384,987, filed on Sep. 8, 2016.

(51) Int. Cl. A61K 38/17; A61K 9/00; A61K 31/351; A61P 19/04; A61K 31/4192; A61K 31/495

(52) U.S. Cl. CPC A61K 38/177; A61K 9/0019; A61K 31/495; A61P 19/04; A61K 31/4192; A61K 31/351

(57) ABSTRACT

The present disclosure relates to anti-fibrotic sialidase-inhibitor compounds and methods of preventing or inhibiting fibrosis using such compounds. The present disclosure also relates to methods of controlling the formation of fibrocytes or their activity using such compounds. The compounds may include both antibodies as well as small molecules. The methods may involve administering the compounds to a patient with or at risk of developing fibrosis in a manner that inhibits at least one sialidase in the patient.

### RELATED APPLICATIONS

This application is a continuation of International Application number PCT/US2017/050435 filed Sep. 7, 2017; which claims priority to U.S. Provisional Application number 62/384,987 filed Sep. 8, 2016, the contents of which are incorporated by reference herein.

### GOVERNMENT RIGHTS

This invention was made with government support under Grant Number HL118507 awarded by the National Institutes of Health. The government has certain rights in the invention.

### TECHNICAL FIELD

The present disclosure relates to anti-fibrotic compounds and methods of preventing or inhibiting fibrosis using such compounds. The compounds may include both antibodies as well as small molecules. The methods may involve administering the compounds to a patient with, or at risk of developing, fibrosis.

### BACKGROUND

Fibrocytes are a specialized type of cell that plays an important role in the body's response to injury and in inflammation. Fibrocytes are formed when they differentiate from CD14+ peripheral blood monocytes. Fibrocytes express markers of both hematopoietic (blood producing) cells (CD45, MHC class II, CD34) and stromal (structural tissue) cells (collagen types I and III and fibronectin). Mature fibrocytes secrete cytokines, extracellular matrix proteins, and pro-angiogenic molecules.

Fibrocytes and the molecules they display extracellularly or release often result in fibrosis, which is the development of scar tissue. Instead of normal wound healing in response to an injury, fibrosis (which may result from inappropriate fibrocyte formation or activity) is harmful if it occurs with too great an extent, for too long, or in an inappropriate location.

As a result, controlling the formation of fibrocytes and their activity in the body may help control harmful fibrosis, thereby avoiding or treating any resulting diseases or disorders.

### SUMMARY

The present disclosure provides a method of preventing or inhibiting fibrosis in a human by administering to a human at least one of Compounds 1-58, disclosed herein, or any combination thereof, in an amount and for a time sufficient to inhibit the activity of at least one human sialidase in the human.

The present disclosure further provides a method of preventing or inhibiting fibrosis in a human by administering to a human at least one of Compounds 1-58, disclosed herein, or any combination thereof, in an amount and for a time sufficient to inhibit the activity or level of Transforming Growth Factor-β1 (TGF-β1) in the human.

The above methods may be combined with one another and alone or in combination may further include one or more of the following additional features, unless clearly mutually exclusive: i) at least one of Compounds 1-58 or any combination thereof may be administered; ii) the activity of at least human neuraminidase 1 (NEU1) in desialylating serum amyloid protein (SAP) may be inhibited; iii) the activity of at least human neuraminidase 2 (NEU2) in desialylating SAP may be inhibited; iv) the activity of at least human neuraminidase 3 (NEU3) in desialylating SAP may be inhibited; v) the activity of at least human neuraminidase 4 (NEU4) in desialylating SAP may be inhibited; vi) the activity of at least one human sialidase on terminal sialic acids with an α(2,6)-linkage may be inhibited; vii) the activity of all wild type human sialidases in the human on terminal sialic acids with an α(2,6)-linkage may be inhibited; viii) the activity of at least one human sialidase on terminal sialic acids with an α(2,3)-linkage may be inhibited; ix) the formation or activation of fibrocytes may be inhibited as a result of inhibition of human sialidase activity on SAP; x) the formation or activation of fibrocytes may be inhibited as a result of inhibition of human sialidase activity on or inhibition of level or activity of TGF-β1; xi) the proliferation or activation of fibroblasts may be inhibited as a result of inhibition of human sialidase activity on level or activity of TGF-β1; xii) at least one of Compounds 1-58 may be administered to the human systemically; xiii) at least one of Compounds 1-58 may be administered to the human locally in an area in which human sialidase activity is abnormally high; xiv) at least one of Compounds 1-58, or any combination thereof, may be administered to the human in an amount and for a time sufficient to inhibit the activity of at least two human sialidases in the human.

The disclosure also provides a method of preventing or inhibiting fibrosis in a human by administering to a human at least one isolated human or humanized monoclonal antibody that binds to the active site of at least one human sialidase wherein the antibody is administered in an amount and for a time sufficient to inhibit the activity of the at least one human sialidase in the human.

The disclosure further provides a method of preventing or inhibiting fibrosis in a human by administering to a human at least one isolated human or humanized monoclonal antibody that binds to the active site of at least one human sialidase wherein the antibody is administered in an amount and for a time sufficient to inhibit the activity of TGF-β1 in the human.

The above methods may be combined with one another and alone or in combination may further include one or more of the following additional features, unless clearly mutually exclusive: i) the activity of at least human NEU1 in desialylating SAP may inhibited; ii) the activity of at least human NEU2 in desialylating SAP may be inhibited; iii) the activity of at least human NEU3 in desialylating SAP may be inhibited; iv) the activity of at least human NEU4 in desialylating SAP may be inhibited; v) the activity of at least one human sialidase on terminal sialic acids with an α(2,6)-linkage may be inhibited; vi) the activity of at least one human sialidase on terminal sialic acids with an α(2,3)-linkage may be inhibited; vii) the activity of all human sialidases in the patient on terminal sialic acids with an α(2,6)-linkage may be inhibited; viii) the formation or activation of fibrocytes may be inhibited as a result of inhibition of human sialidase activity on SAP; ix) at least two isolated human or humanized monoclonal antibodies that bind to the active site of at least one human sialidase may be administered in an amount and for a time sufficient to inhibit the activity of the at least one human sialidase in the human.

In addition any the above methods directed to administering at least one of Compounds 1-58, or any combinations thereof and the methods direct to administering at least one isolated human or humanized monoclonal antibody may be combined to administer both a compound and an antibody to the patient either simultaneously, such that both are in the circulation at the same time, or in series.

### BRIEF DESCRIPTION OF THE DRAWINGS

The figures form part of the present specification and are included to further demonstrate certain aspects of the present invention. The figures are not intended to and should not be interpreted to encompass the entirety of the invention. In addition, different aspects of the invention are often illustrated as separate figures for clarity; these aspects may be combined with one another unless clearly not compatible.

FIG. 1A is a not-to-scale schematic diagram of feedback pathways between sialidases and fibrosis.

FIGS. 1B-1E are graphs of interluekin-6 (IL-6) levels in supernatants of peripheral blood mononuclear cells (PBMC) incubated in serum free or serum-containing medium with indicated concentrations of recombinant NEU3. In FIG. 1B, the human PBMCs were incubated in serum free medium for two days. In FIG. 1C, the human PBMCs were incubated in serum free medium for five days. In FIG. 1D, the human PBMCs were incubated in serum-containing medium for two days. In FIG. 1E, the human PBMCs were incubated in serum-containing medium for five days. Values are mean±SEM, n=3. *p<0.05, **p<0.01 compared to no added NEU3 (t-tests).

FIGS. 1F-1G are graphs of NEU3 levels in monocytes (FIG. 1F) and lymphocytes (FIG. 1G) incubated with the indicated concentrations of recombinant human IL-6. Graphs show the median fluorescence for monocytes (FIG. 1G) or lymphocytes (FIG. 1F) identified by forward- and side-scatter. Values are mean±SEM, n=3. *p<0.05, **p<0.01, ***p<0.001 for NEU3 compared to no added IL-6 (2-way ANOVA, Sidak's multiple comparison test). Values for the other sialidases were not significantly different from control.

FIGS. 2A-2E show protein sialylation in human lungs with and without pulmonary fibrosis.

FIG. 2A is a set of photomicrographs of human lung sections stained with biotinylated Sambucus nigra lectin (SNA) to detect α(2,6)-linked terminal sialic acids or biotinylated peanut agglutinin (PNA) to detect desialylated polysaccharides. ILD <50% FEV1 designates lung sections from a pulmonary fibrosis patient with poor lung function. COPD >80% FEV1 designates lung sections from a chronic obstructive pulmonary disease patient with relatively normal lung function. Bar is 0.2 mm.

FIG. 2B is a graph of quantified lectin (SNA) staining for human lung sections as in FIG. 2A. Values are mean±SEM, n=4; ** indicates p<0.005, ****p<0.0001 (t-test).

FIG. 2C is a set of photomicrographs of sections of human lungs stained with anti-NEU1 antibodies, anti-NEU2 antibodies, anti-NEU3 antibodies, or anti-NEU4 antibodies. Pulmonary Fibrosis designates lung sections from a pulmonary fibrosis patient with poor lung function. COPD designates lung sections from a chronic obstructive pulmonary disease patient with relatively normal lung function. Outside image bars are 0.2 mm. Inset bars are 0.1 mm. Images are representative of four patients per group.

FIG. 2D is a graph of quantified anti-sialidase antibody staining for human lung sections as in FIG. 2C. Values are mean±SEM, n=4; ** indicates p<0.005, ****p<0.0001 (t-test).

FIG. 2E is a photomicrograph of a section of human lung as in FIG. 2C, in which a different result using anti-NEU1 antibodies was obtained. Bar is 0.2 mm.

FIG. 3A is a photomicrograph of a section of normal human heart stained with anti-NEU3 antibody. Positive staining is red. Bar is 0.1 mm.

FIG. 3B is a photomicrograph of a section of a fibrotic region of a human heart from a patient with dilated cardiomyopathy stained with anti-NEU3 antibody. Positive staining is red. Bar is 0.1 mm.

FIG. 4A is a photomicrograph of a section of normal human liver stained with anti-NEU3 antibody. Positive staining is red. Bar is 0.1 mm.

FIG. 4B is a photomicrograph of a section of a human liver from a patient with steatosis and fibrosis stained with anti-NEU3 antibody. Positive staining is red. Bar is 0.1 mm.

FIG. 5 is a graph of the effects of sialidases on fibrocyte formation in humans and the effects of DANA and SAP in the presence of sialidases on fibrocyte formation in humans. Human PBMCs were incubated in serum-free medium in the absence of sialidase or in the presence of recombinant human NEU1 (sialidase 1), NEU2 (sialidase 2), NEU3 (sialidase 3), or NEU4 (sialidase 4). Cells were also incubated in a control solution or in the presence of the sialidase inhibitor N-Acetyl-2,3-dehydro-2-deoxyneuraminic acid (DANA) or human SAP. Fibrocytes were counted after 5 days. Numbers as a percent of the no-sialidase control are reported in the graph. Values are mean±standard error of the mean (SEM), n=3 blood donors per group. * designates p<0.05; ** designates p<0.01. X indicates p<0.05 compared to control. No fibrocytes were detected in the cultures with SAP and no sialidase or SAP and sialidase 1.

FIG. 6A is a set of photomicrographs of A549 cells cultured in the presence or absence of TGF-β1 and stained for the indicated sialidase. Positive staining is red. Bar is 0.2 mm.

FIG. 6B is a set of photomicrographs of human small airway epithelial cells cultured in the presence or absence of TGF-β1 and stained for the indicated sialidase. Positive staining is red. Bar is 0.2 mm.

FIG. 6C is a set of photomicrographs of human pulmonary fibroblast cells cultured in the presence or absence of TGF-β1. Bar is 0.2 mm.

FIG. 6D is a set of photomicrographs of human PBMC cultured in the presence or absence of TGF-β1 and stained for the indicated sialidase. Positive staining is red. Bar is 0.2 mm.

FIG. 7A is a set of photomicrographs of human PBMC cultured with or without recombinant human sialidases and stained for TGF-β1. Positive staining appears pink and counter staining is blue. Bar is 0.1 mm.

FIG. 7B is a graph presenting positively stained macrophages from images as in FIG. 7A as a percent of total cells. Values are mean±SEM, n=3. * p<0.05, ** p<0.005 (t-test).

FIG. 7C is a graph of quantified TGF-β1 staining for PBMC as in FIG. 7A. Values are mean±SEM, n=3. *p<0.05 (t-test).

FIG. 7D is a graph of TGF-β1 ELISA results for human PBMC incubated with or without recombinant human sialidases, DANA, or oseltamivir. Values are mean±SEM, n=7. * p<0.05, **p<0.01 (t tests).

FIG. 7E is a set of photomicrographs of human PBMC cultured in serum free media with or without recombinant human sialidases, then stained with antibodies against NEU1, NEU2, NEU3, or NEU4. Bar is 0.2 mm.

FIG. 8A is a set of photomicrographs of sections of mouse lungs stained with biotinylated Maackia amurensis lectin II (MAL II) to detect α(2,3)-linked sialic acids or PNA to detect desialylated polysaccharides. The sections labeled Bleo are from mice treated with bleomycin to induce lung fibrosis. The sections labeled Saline are from mice treated with only saline. Lung sections were taken 21 days after treatment.

FIG. 8B is a graph of quantified lectin (MAL II) staining for mouse lung sections as in FIG. 8A. Values are mean±SEM, n=3; ** indicates p<0.005, ****p<0.0001 (t-test).

FIG. 8C is a set of photomicrographs of sections of mouse lungs stained with anti-NEU1 antibodies, anti-NEU2 antibodies, anti-NEU3 antibodies, or anti-NEU4 antibodies. The sections labeled Bleomycin are from mice treated with bleomycin to induce lung fibrosis. The sections labeled Saline are from mice treated with only saline. Lung sections were taken 21 days after treatment. Outside image bars are 0.2 mm. Inset bars are 0.1 mm. Images are representative of three mice per group.

FIG. 8D is a graph of quantified anti-sialidase antibody staining for mouse lung sections as in FIG. 8C. Values are mean±SEM, n=4; * indicates p<0.05, ****p<0.0001 (t-test).

FIG. 8E is two Western blots of bronchoalveolar lavage fluid (BAL fluid) from mouse lungs. BAL was obtained 21 days after treatment with bleomycin to induce lung fibrosis (Bleo), or treatment with saline (S). Western blots were stained with either MAL II to detect α(2,3)-linked sialic acids or PNA to detect desialylated polysaccharides. * indicates where a band would appear for sialylated proteins in the Bleo sample stained with MAL II. The arrow indicates where desialylated proteins appear in the S and Bleo samples stained with PNA. Molecular masses in kDa are at left. Images are representative of three mice per group.

FIG. 8F is a Western blot of BAL from mouse lungs. BAL was obtained 21 days after treatment with bleomycin to induce lung fibrosis or saline. Three samples for each group are included. The Western blot was stained with anti-NEU3 antibodies. Molecular masses in kDa are at left.

FIG. 8G is a graph of quantified anti-NEU3 antibody staining of the right Western blot of FIG. 8F. Values are expressed in percent relative density of black bands. Values are mean±SEM, n=3; * indicates p<0.05 (t-test).

FIG. 8H is a graph of ELISA-quantified NEU1 in the lungs of bleomycin-treated and saline-treated mice. Values are mean±SEM, n=3; * indicates p<0.05 (t-test).

FIG. 8I is a graph of ELISA-quantified NEU2 in the lungs of bleomycin-treated and saline-treated mice. Values are mean±SEM, n=3; * indicates p<0.05 (t-test).

FIG. 8J is a graph of ELISA-quantified NEU3 in the lungs of bleomycin-treated and saline-treated mice. Values are mean±SEM, n=3; ** indicates p<0.005 (t-test).

FIG. 8K is a graph of ELISA-quantified NEU4 in the lungs of bleomycin-treated and saline-treated mice.

FIG. 8L is a Western blot of lung tissue lysate from a saline-treated mouse (Sal) and a bleomycin-treated mouse (Bleo) stained for NEU3 (upper panel). Aliquots of the samples were run on a SDS-PAGE gel and stained with Coomassie brilliant blue (CB) (lower panel) to show total protein. The positions of molecular mass standards in kDa are at left. Images are representative of 3 mice per treatment group.

FIG. 8M is a graph of quantified protein staining of Western blots as FIG. 8L. Values are mean±SEM, n=3; * indicates p<0.05 (t-test).

FIGS. 9A-9J show the effects of sialidase inhibitors on fibrosis in mice. For FIGS. 9B-9E and 9G-9J, values are mean±SEM. n=3 mice per group. For FIGS. 9B-9D and 9G-9J, * designates p<0.05, ** designates p<0.01 as determined by 1-way ANOVA, Tukey's test.

FIG. 9A is a set of photomicrographs of mouse lung sections stained for collagen with Sirius red. Bar is 0.2 mm. The sections labeled Bleo are from mice treated with bleomycin to induce lung fibrosis. The section labeled Saline is from a mouse treated with only saline. Lung sections were taken 21 days after treatment. Sections also labeled DANA are from mice also treated with the sialidase inhibitor DANA starting at day 1. Sections also labeled Oseltamivir are from mice treated with the sialidase inhibitor oseltamivir starting at day 1.

FIG. 9B is a graph quantifying Sirius red staining results for mice in the same groups as in FIG. 9A.

FIG. 9C is a graph of the total number of cells recovered from BAL 21 days after treatment with saline, bleomycin (Bleo), bleomycin with DANA (Bleo+DANA), or bleomycin and oseltamivir (Bleo+Oseltamivir) in the same groups as in FIG. 9A.

FIG. 9D is a graph of the total number of either CD11b+ cells or CD11c+ cells recovered from BAL 21 days after treatment with saline, bleomycin (Bleo), bleomycin with DANA (Bleo+DANA) or bleomycin and oseltamivir (Bleo+Oseltamivir) in the same groups as in FIG. 9A.

FIG. 9E is a graph of the total protein levels in BAL 21 days after treatment with saline, bleomycin (Bleo), bleomycin with DANA (Bleo+DANA) or bleomycin and oseltamivir (Bleo+Oseltamivir) in the same groups as in FIG. 9A. Values are BAL protein concentration x collected BAL volume.

FIG. 9F is a set of photomicrographs of mouse lung sections stained for collagen with Sirius red. Bar is 0.2 mm. The sections labeled Bleo are from mice treated with bleomycin to induce lung fibrosis. The section labeled Saline is from a mouse treated with only saline. Lung sections were taken 21 days after treatment. Sections also labeled DANA are from mice also treated with DANA starting at day 10. Sections also labeled Oseltamivir are from mice also treated with oseltamivir starting at day 10.

FIG. 9G is a graph quantifying Sirius red staining results for mice in the same groups as in FIG. 9F.

FIG. 9H is a graph of the total number of cells recovered from BAL 21 days after treatment with saline, bleomycin (Bleo), bleomycin with DANA (Bleo+DANA), or bleomycin and oseltamivir (Bleo+Oseltamivir) in the same groups as in FIG. 9F.

FIG. 9I is a graph of the total number of CD11b+ cells recovered from BAL 21 days after treatment with saline, bleomycin (Bleo), bleomycin with DANA (Bleo+DANA) or bleomycin and oseltamivir (Bleo+Oseltamivir) in the same groups as in FIG. 9F.

FIG. 9J is a graph of the total protein levels in BAL 21 days after treatment with saline, bleomycin (Bleo), bleomycin with DANA (Bleo+DANA) or bleomycin and oseltamivir (Bleo+Oseltamivir) in the same groups as in FIG. 9F. Values are BAL protein concentration×collected BAL volume. 0.055 indicates p=0.055.

FIG. 9K is a set of photomicrographs of sections of lung tissue corresponding to FIG. 9F stained with antibodies against NEU1, NEU2, NEU3 and NEU4. Bar is 0.2 mm.

FIG. 9L is a graph quantifying anti-NEU1 staining results for mice in the same group as FIG. 9K Values are mean±SEM, n=3; ** indicates p<0.005, ***p<0.001 (t-test).

FIG. 9M is a graph quantifying anti-NEU2 staining results for mice in the same group as FIG. 9K Values are mean±SEM, n=3; ** indicates p<0.005, ***p<0.001 (t-test).

FIG. 9N is a graph quantifying anti-NEU3 staining results for mice in the same group as FIG. 9K Values are mean±SEM, n=3; ** indicates p<0.005, ***p<0.001 (t-test).

FIG. 9O is a graph quantifying anti-NEU4 staining results for mice in the same group as FIG. 9K Values are mean±SEM, n=3; ** indicates p<0.005, ***p<0.001 (t-test).

FIG. 10 is a graph of the sialic acid content of pieces of mouse lung at day 21 after oropharyngeal saline or oropharyngeal bleomycin (Bleo) treatment. Values are mean±SEM, n=3.

FIG. 11A is a set of photomicrographs of sections of lung tissue stained for TGF-β1 from mice treated with bleomycin or saline, then with saline, DANA, or oseltamivir.

FIG. 11B is a graph quantifying TGF-β1 staining results for mice as in FIG. 11A. Values are mean±SEM, n=3 mice per group. * indicates p<0.05 (t-test).

### DETAILED DESCRIPTION

The present disclosure relates to anti-fibrotic compounds and methods of preventing or inhibiting fibrosis using such compounds. The compounds and methods may also prevent or inhibit fibrocyte formation, including proliferation, or fibrocyte activity, including activation, that may give rise to fibrosis.

The compounds may include both antibodies as well as small molecules. The compounds may inhibit a sialidase, particularly a human sialidase.

The methods may involve administering the compounds to a patient with or at risk of developing fibrosis or with abnormal fibrocyte formation, including proliferation, or activity, including activation. The amount of compound administered, the mode of administration, the dose, and frequency of any repetitions may vary depending on the compound and effect to be achieved.

### Sialidases and Fibrosis

A protein with an attached polysaccharide is referred to as a glycosylated protein. Many of the polysaccharides on glycosylated proteins have a sialic acid monosaccharide, particularly at the end distal to the protein (referred to as a terminal sialic acid). Sialidases (also called neuraminidases) are enzymes that remove sialic acid from polysaccharides found on glycosylated proteins.

Sialidases are used by a wide variety of organisms, including harmful viruses and bacteria. Mammals have four sialidases, designated NEU1, NEU2, NEU3, and NEU4.

NEU1 is generally expressed at higher levels than the other three sialidases and it is expressed in most tissues, with higher levels in the lung and airway epithelial cells than in most other cells. NEU1 is located in lysosomes and in the plasma membrane, with its catalytic domain outside the cell. NEU1 preferentially desialylates terminal sialic acids with α(2,6)-linkages and to a lesser extent α(2,3)-linkages, with also some activity for α(2,8)-linkages.

NEU2 is a soluble, cytosolic enzyme. NEU2 preferentially desialylates terminal sialic acids with α(2,3)-linkages, α(2,6)-linkages, and α(2,8)-linkages.

NEU3 is a plasma membrane-associated sialidase. NEU3 preferentially desialylates terminal sialic acids with α(2,3)-linkages, α(2,6)-linkages, and α(2,8)-linkages.

NEU4 has two isoforms, one with a mitochondrial localization, while the other is associated with intracellular membranes. NEU4 preferentially desialylates terminal sialic acids with α(2,3)-linkages, α(2,6)-linkages, and α(2,8)-linkages.

Variants of these four sialidases are known in humans and in other mammals. In particular, variants with single nucleotide polymorphisms (SNPs) are known.

Sialidases are associated with inflammation in mammals and inflammation increases the presence of sialidases. Thus, a positive feedback loop exists between inflammation and sialidases in mammals.

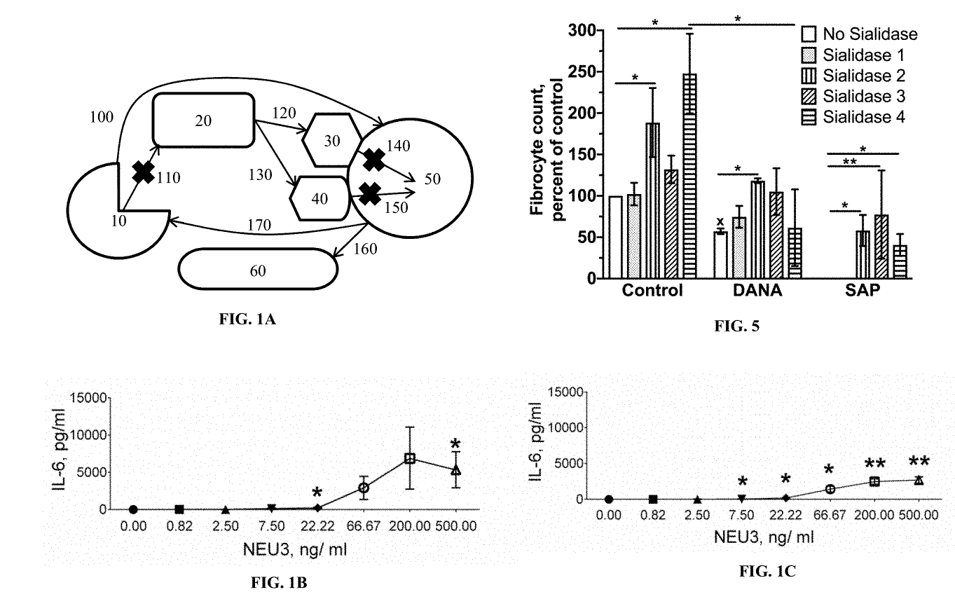

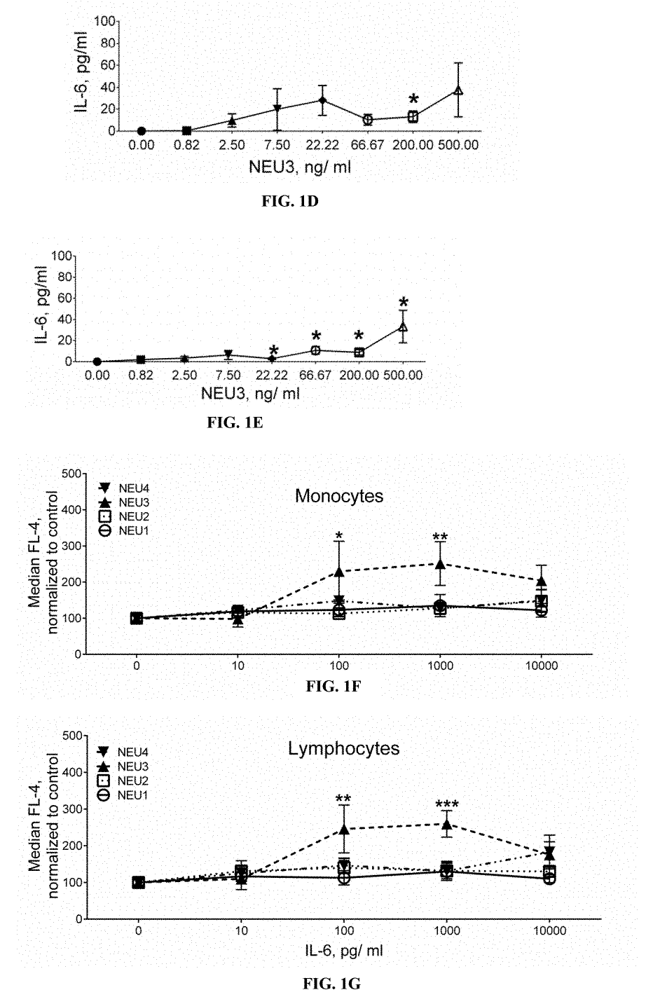

The present disclosure exploits a newly discovered feedback pathway between sialidases and fibrosis in mammals, including humans. At least a portion of this pathway is illustrated in FIG. 1A.

SAP 20, when in its normal glycosylated form, binds to DC-SIGN 30 via binding pathway 120. This causes DC-SIGN 30 to inhibit activation of profibrotic innate immune system cell 50 via inhibition pathway 140.

SAP 20, when in its normal glycosylated from, may also bind to Fcγ Receptor 1 (FcγR1) 40 via binding pathway 130. This causes FcγR1 to inhibit activation of profibrotic innate immune system cell 50 via inhibition pathway 150.

In the absence of inhibition, profibrotic innate immune system cell 50 may secrete cytokines, including TGF-β, Tumor Necrosis Factor α (TNFα), Interleukin-4 (IL-4), IL-6, and IL-13. These cytokines act via pathways 160 to cause the formation or activation of fibroblasts 60. Fibroblasts 60 may then go on to cause fibrosis.

In addition, TGF-β1 secreted by innate immune system cell 50, present internally in innate immune system cell 50 in increased amounts, or otherwise present in the extracellular environment may increase the expression of sialidase 10.

When SAP 20 binds to either DC-SIGN 30, FcγR1 40, or both, causing inhibition of profibrotic innate immune system cell 50, then cytokines are not secreted and formation or activation of fibroblasts 60 does not occur.

Sialidase 10 may act via general pathway 100 to promote the secretion of cytokines by profibrotic innate immune system cell 50 and the formation or activation of fibroblasts 60.

Pathway 100 may include a variety of subpathways, but in at least one subpathway, sialidase 10 acts upon glycosylated SAP 20 to cleave the terminal sialic acid from the SAP polysaccharide. Sialidase 10 may be NEU1, which is able to cleave the type of sialic acid linkage present on glycosylated SAP. It may also be NEU2, NEU3, NEU4, or a combination of any sialidases.

Cleaving the sialic acid from glycosylated SAP 20 is an inhibitory process, as illustrated by pathway 110. SAP 20 lacking sialic acid is not able to effectively bind to DC-SIGN 30 or FcγR1 40 via pathway 120 or pathway 130, and thus SAP 20 is not able to inhibit profibrotic innate immune system cell 50 via inhibitory pathways 140 and 150. This leaves profibrotic innate immune system cell 50 free to promote the formation or activation of fibroblasts 60.

In addition to promoting the formation or activation of fibroblasts 60, profibrotic innate immune system cell 50, or possibly fibroblasts 60, also act via pathway 170 to further active sialidase 10. Thus, via pathways 100 and 170, fibrocyte formation or activation and sialidase activity form a positive feedback loop.

This positive feedback loop may be beneficial in some biological contexts, but it may also contribute to runaway fibrosis in fibrosing diseases and disorders. The present disclosure, therefore, provides compounds to disrupt this positive feedback look and methods of using them to prevent or control the damaging effects of fibrocyte formation or activation.

As an example of this positive feedback loop, NEU3 causes human PBMC to accumulate IL-6, which in turn induces human PBMC to accumulate NEU3.

As another example, sialidases cause profibrotic innate immune system cells or other cells to secrete TGF-β1 into the extracellular environment or increase internal amounts, which then increase sialidase expression.

As a third example of this positive feedback loop, one sialidase, such as NEU2, may increase expression of another sialidase, such as NEU3.

These example feedback loops may all be present in the same cells or biological system and other feedback loops may further be present as well.

### Sialidase Inhibitors

The present disclosure includes anti-fibrotic sialidase inhibitors, particularly human sialidase inhibitors, and their use in preventing or inhibiting fibrosis.

Human sialidase inhibitors may inhibit the enzymatic activity of all human sialidases, a subset of human sialidases, or one human sialidase, all in wild type form alone or also including one or more active variants. In particular, sialidase inhibitors may inhibit the enzymatic activity of at least human NEU1 alone, human NEU2 alone, human NEU3 alone, or human NEU4 alone. Enzymatic activity may be defined as inhibited if the rate measured by the Michaelis-Menten equation in an in vitro assay using a substrate with a terminal sialic acid is inhibited by at least 50%. More specifically, a human sialidase inhibitor may be a compound that inhibits the rate of at least one human sialidase by at least 50% as measured by the Michaelis-Menten equation in an in vitro assay using the fluorometric substrate 4MU-NANA [2′-(4-Methylumbelliferyl)-α-D-N-acetylneuraminic acid.

Human sialidase inhibitors may also inhibit TGF-β1 activity or level, which may interrupt the positive feedback loop described in FIG. 1A and thus also prevent or inhibit fibrosis.

Small Molecule Sialidase Inhibitors

Small molecule sialidase inhibitors, particularly human sialidase inhibitors, may include the compound having the following structural formula:

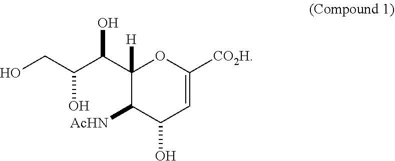 [SMILES: CC(=O)N[C@H]1[C@H]([C@H](O)[C@H](O)CO)OC(C(=O)O)=C[C@@H]1O]

Compound 1, also known as DANA, inhibits human NEU1 with an inhibitory concentration of 50% of enzymatic activity (IC₅₀) of 143 μM. DANA inhibits human NEU2 with an IC₅₀ of 43 μM, human NEU3 with an IC₅₀of 61 μM, and human NEU 4 with an IC₅₀ of 74 μM.

Small molecule sialidase inhibitors may also include compounds have the following general structural formula:

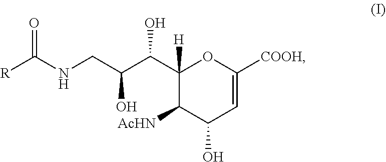

in which R is a group as presented in Table 1. Table 1 also includes IC₅₀ concentrations for the various R groups for the four human sialidases.

TABLE 1
R groups for Formula I and IC₅₀ Concentrations

| | | NEU1 IC₅₀, | NEU2 IC₅₀, | NEU3 IC₅₀, | NEU4 IC₅₀, |
|---|---|---|---|---|---|
| Compound # | R | μM | μM | μM | μM |
| 2 | Methyl | 58 | >1000 | >1000 | 580 |
| 3 | Phenyl | 13 | 865 | 320 | 810 |
| 4 | Cyclopentyl | 135 | >1000 | >1000 | >1000 |
| 5 | Cyclopropyl | 680 | >1000 | >1000 | 825 |
| 6 | Propyl | 32 | >1000 | >1000 | 923 |
| 7 | 2-Methylpropyl | 565 | >1000 | >1000 | >1000 |
| 8 | n-Butyl | 10 | >1000 | >1000 | >1000 |

Small molecule sialidase inhibitors may also the compound having the following structural formula:

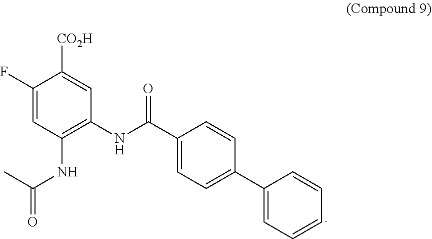 [SMILES: CC(=O)Nc1cc(F)c(C(=O)O)cc1NC(=O)c1ccc(-c2ccccc2)cc1]

Compound 9 inhibits human NEU2 with an IC₅₀ of 0.55±0.12 mM.

Small molecule sialidase inhibitors may also include a compound having the following structural formula:

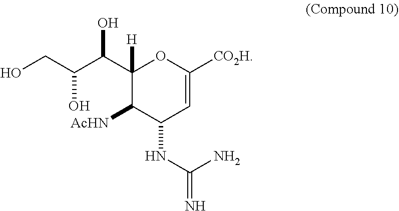 [SMILES: CC(=O)N[C@H]1[C@H]([C@H](O)[C@H](O)CO)OC(C(=O)O)=C[C@@H]1NC(=N)N]

Compound 10, also known as zanamivir ((2R,3R,4S)-4-guanidino-3-(prop-1-en-2-ylamino)-2-((1R,2R)-1,2,3-trihydroxypropyl)-3,4-dihydro-2H-pyran-6-carboxylic acid) inhibits human NEU2 with an inhibitory constant (Kᵢ) of 0.017 mM.

Small molecule sialidase inhibitors may also include a compound having the following structural formula:

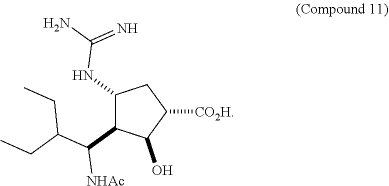 [SMILES: CCC(CC)C(NC(C)=O)[C@@H]1[C@H](O)[C@@H](C(=O)O)C[C@H]1NC(=N)N]

Compound 11, also known as peramivir ((1S,2S,3S,4R)-3-[(1S)-1-acetamido-2-ethyl-butyl]-4-(diaminomethylideneamino)-2-hydroxy-cyclopentane-1-carboxylic acid) inhibits human NEU2 with a Kᵢ of 0.33 mM.

Small molecule sialidase inhibitors may also include a compound having the following structural formula:

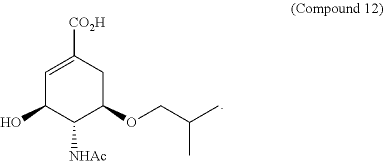 [SMILES: CC(=O)N[C@@H]1[C@@H](O)C=C(C(=O)O)C[C@H]1OCC(C)C]

Compound 12 inhibits human NEU2 with a Kᵢ of 0.88 mM.

Small molecule sialidase inhibitors may also include a compound having the following structural formula:

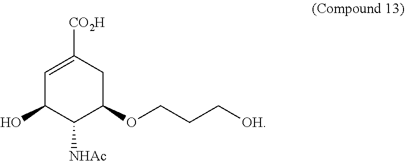 [SMILES: CC(=O)N[C@@H]1[C@@H](O)C=C(C(=O)O)C[C@H]1OCCCO]

Compound 13 inhibits human NEU2 with a Kᵢ of 0.74 mM.

Small molecule sialidase inhibitors may also include a compound having the following structural formula:

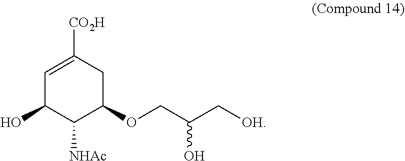 [SMILES: CC(=O)N[C@@H]1[C@@H](O)C=C(C(=O)O)C[C@H]1OCC(O)CO]

Compound 14 inhibits human NEU2 with a Kᵢ of 1.4 mM.

Small molecule sialidase inhibitors may also include a compound having the following general structural formula:

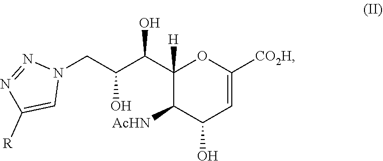

in which R is a group as presented in Table 2. Table 2 also includes IC₅₀ concentrations for the various R groups for human NEU3.

TABLE 2
R groups for Formula II and IC₅₀ Concentrations

| Compound | | | NEU3 IC₅₀, |
|---|---|---|---|
| # | R name | R structure | μM |
| 15 | H | n/a | 70 ± 20 |
| 16 | phenyl | 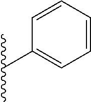 [SMILES: *c1ccccc1] | 20 ± 10 |
| 17 | hexyl | 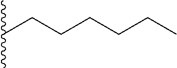 [SMILES: *CCCCCC] | 23 ± 4 |
| 18 | ethoxy | 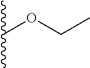 [SMILES: *OCC] | 300 ± 200 |
| 19 | triazole | 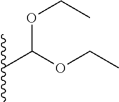 [SMILES: *C(OCC)OCC] | 130 ± 20 |
| 20 | phenoxymethyl | 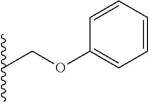 [SMILES: *COc1ccccc1] | 45 ± 3 |
| 21 | hydroxymethyl | 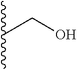 [SMILES: *CO] | 300 ± 400 |
| 22 | hydroxyethyl | 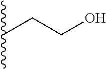 [SMILES: *CCO] | 400 ± 600 |
| 23 | hydroxypropyl | 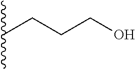 [SMILES: *CCCO] | 500 ± 200 |
| 24 | | 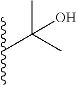 [SMILES: *C(C)(C)O] | 300 ± 500 |

Small molecule sialidase inhibitors may also include a compound having the following structural formula:

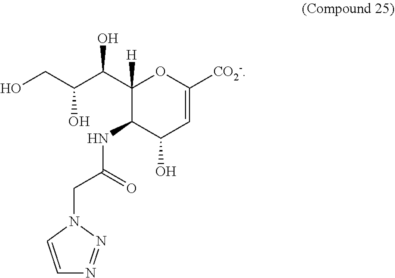

Compound 25 inhibits human NEU3 with a Kᵢ of 21±8 μM.

Small molecule sialidase inhibitors may also include a compound having the following general structural formula:

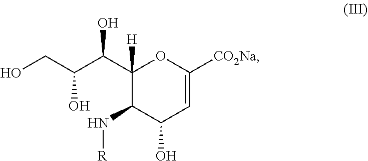

in which R is a group as presented in Table 3. Table 3 also includes IC₅₀ concentrations for the various R groups for human NEU2.

TABLE 3
R groups for Formula III and IC₅₀ Concentrations

| | | | IC₅₀ for |
|---|---|---|---|
| Com- | | | NEU2, |
| pound # | R | Full name | μM |
| 38 | -Ac | 5-Acetamido-2,6-anhydro-3,5-dideoxy-D- | 18 ± 1 |
| | | glycero-D-galactonon-2-enonic acid | |
| 39 | -Gc | 2,6-Anhydro-3,5-dideoxy-5-glycolylamido-D- | 34 ± 4 |
| | | glycero-D-galactonon-2-enonic acid | |
| 40 | -AcN₃ | 2,6-Anhydro-5-(2-azidoacetamido)-3,5-dideoxy- | 8.3 ± 1 |
| | | D-glycero-D-galacto-non-2-enonic acid | |

Small molecule sialidase inhibitors may also include a compound having the following structural formula:

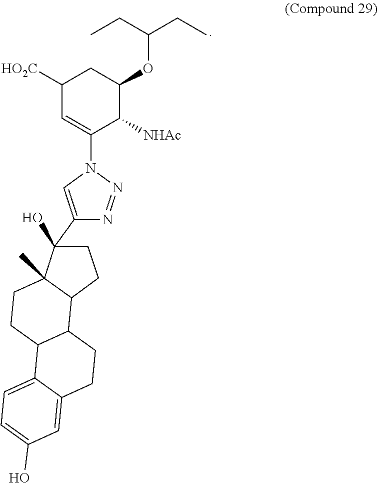 [SMILES: CCC(CC)O[C@@H]1CC(C(=O)O)C=C(n2cc([C@]3(O)CCC4C5CCc6cc(O)ccc6C5CC[C@@]43C)nn2)[C@H]1NC(C)=O]

Compound 29 inhibits human NEU3 with an IC₅₀ of 350±100 μM and it inhibits human NEU 4 with an IC₅₀of 800±400 μM.

Small molecule sialidase inhibitors may also include a compound having the following structural formula:

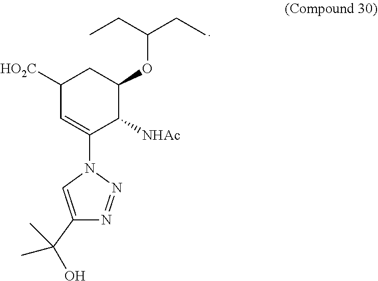 [SMILES: CCC(CC)O[C@@H]1CC(C(=O)O)C=C(n2cc(C(C)(C)O)nn2)[C@H]1NC(C)=O]

Compound 30 inhibits human NEU3 with an IC₅₀ of 640±210 μM.

Small molecule sialidase inhibitors may also include a compound having the following general structural formula:

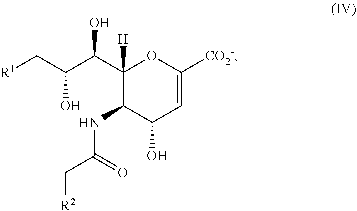

in which R¹ and R² are the groups as presented in Table 4. Table 4 also includes IC₅₀ concentrations for the various R groups for human NEU2.

TABLE 4
R groups for Formula IV and IC₅₀ Concentrations

| Compound | | | IC₅₀, |
|---|---|---|---|
| # | R¹ | R² | μM |
| 31 | —OH | —H | 32 ± 6 |
| 32 | —N₃ | —H | 31 ± 6 |
| 33 | —N₃ | —OH | (1.9 ± 0.7) × 10² |
| 34 | —N₃ | —N₃ | 13 ± 3 |
| 35 | —OCH₃ | —H | (4.1 ± 0.6) × 10² |
| 36 | 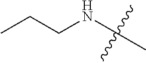 [SMILES: *NCCC] | —H | (2.4 ± 0.8) × 10³ |

Small molecule sialidase inhibitors may also include a compound having the following structural formula:

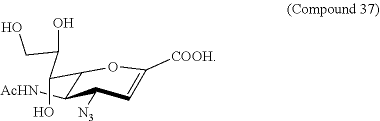

Compound 37 inhibits human NEU1 with an IC₅₀ of 360±50 μM, human NEU2 with an IC₅₀ of 59±13 μM, human NEU3 with an IC₅₀ of 54±5 μM, and human NEU4 with an IC₅₀ of 1000±60 μM.

Small molecule sialidase inhibitors may also include a compound having the following structural formula:

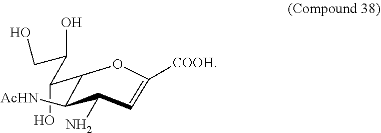

Compound 38 inhibits human NEU2 with an IC₅₀ of 44±3 μM, human NEU3 with an IC₅₀ of 180±20 μM, and human NEU4 with an IC₅₀ of 720±70 μM.

Small molecule sialidase inhibitors may also include a compound having the following structural formula:

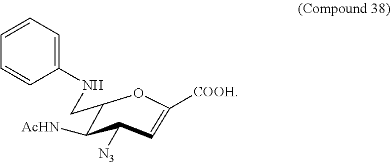

Compound 39 inhibits human NEU 2 with an IC₅₀ of 131±13 μM, human NEU3 with an IC₅₀ of 440±300 μM, and human NEU4 with an IC₅₀ of 300±20 μM.

Small molecule sialidase inhibitors may also include a compound having the following structural formula:

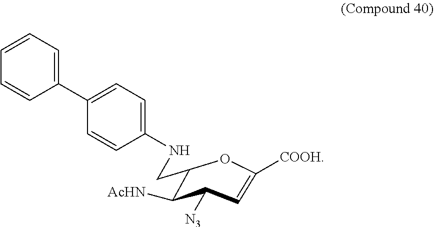

Compound 40 inhibits human NEU2 with an IC₅₀ of 74±4 μM, human NEU3 with an IC₅₀ of 50±30 μM, and human NEU4 with an IC₅₀ of 210±10 μM.

Small molecule sialidase inhibitors may also include a compound having the following structural formula:

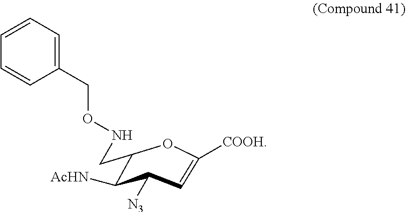

Compound 41 inhibits human NEU2 with an IC₅₀ of 920±200 μM and human NEU3 with an IC₅₀ of 24±2 μM.

Small molecule sialidase inhibitors may also include a compound having the following structural formula:

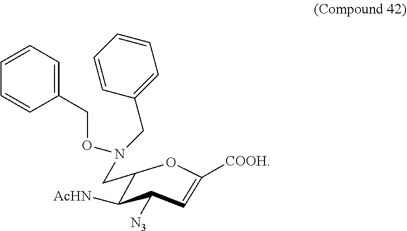

Compound 42 inhibits human NEU2 with an IC₅₀ of 173±50 μM, human NEU3 with an IC₅₀ of 24±11 μM, and human NEU4 with an IC₅₀ of 350±180 μM.

Small molecule sialidase inhibitors may also include a compound having the following structural formula:

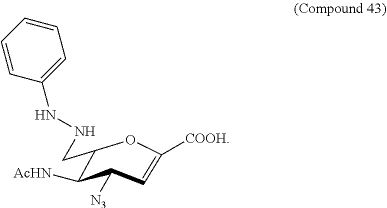

Compound 43 inhibits human NEU2 with an IC₅₀ of 40±7 μM and human NEU3 with an IC₅₀ of 20±8 μM.

Small molecule sialidase inhibitors may also include a compound having the following structural formula:

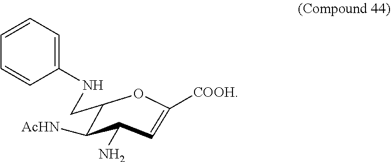

Compound 44 inhibits human NEU2 with an IC₅₀ of 800±30 μM and human NEU3 with an IC₅₀ of 540±30 μM.

Small molecule sialidase inhibitors may also include a compound having the following structural formula:

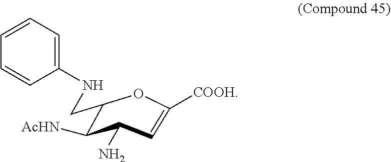

Compound 45 inhibits human NEU2 with an IC₅₀ of 100±13 μM and human NEU3 with an IC₅₀ of 370±80 μM.

Small molecule sialidase inhibitors may also include a compound having the following structural formula:

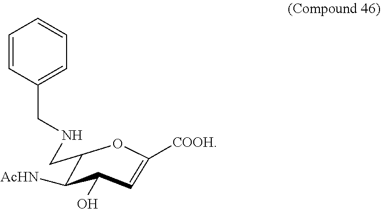

Compound 46 inhibits human NEU2 with an IC₅₀ of 86±17 μM.

Small molecule sialidase inhibitors may also include a compound having the following structural formula:

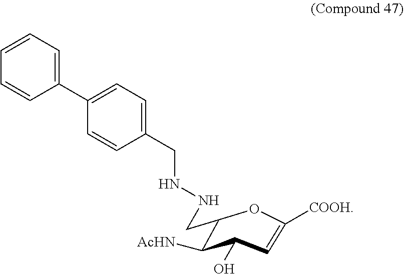

Compound 47 inhibits human NEU2 with an IC₅₀ of 67±18 μM, human NEU3 with an IC₅₀ of 70±20 μM, and human NEU4 with an IC₅₀ of 200±20 μM.

Small molecule sialidase inhibitors may also include a compound having the following structural formula:

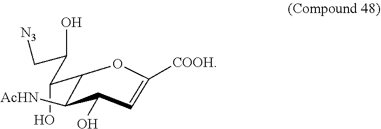

Compound 48 inhibits human NEU1 with an IC₅₀ of 620±10 μM, human NEU2 with an IC₅₀ of 240±20 μM, human NEU3 with an IC₅₀ of 19.7±2.3 μM, and human NEU4 with an IC₅₀ of 60±20 μM.

Small molecule sialidase inhibitors may also include a compound having the following structural formula:

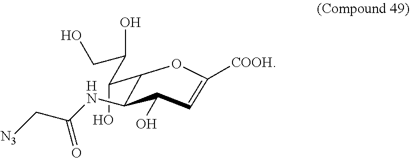

Compound 49 inhibits human NEU1 with an IC₅₀ of 29.0±0.5 μM, human NEU2 with an IC₅₀ of 37±5 μM, human NEU3 with an IC₅₀ of 4.7±0.3 μM, and human NEU4 with an IC₅₀ of 4.5±0.1 μM.

Small molecule sialidase inhibitors may also include a compound having the following structural formula:

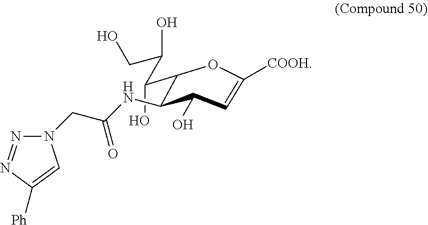

Compound 50 inhibits human NEU2 with an IC₅₀ of 9±1 μM and human NEU4 with an IC₅₀ of 250±40 μM.

Small molecule sialidase inhibitors may also include a compound having the following general structural formula:

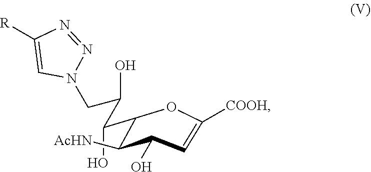

in which R is a group as presented in Table 5. Table 5 also includes IC₅₀ concentrations for the various R groups for human sialidases.

TABLE 5
R groups for Formula IV and IC₅₀ Concentrations

| | | NEU1 IC₅₀, | NEU2 IC₅₀, | NEU3 IC₅₀, | NEU4 IC₅₀, |
|---|---|---|---|---|---|
| Compound # | R | μM | μM | μM | μM |
| 51 | -Phenyl | 200 ± 30 | 250 ± 70 | 4.6 ± 0.2 | 2.3 ± 0.2 |
| 52 | —OCH₂CH₃ | 170 ± 20 | 130 ± 20 | 90 ± 10 | 3.6 ± 0.3 |
| 53 | —CH₂OPhenyl | >1000 | 800 ± 70 | 5.5 ± 0.6 | 1.7 ± 0.2 |
| 54 | —CH₂OH | >1000 | >1000 | 80 ± 10 | 0.16 ± 0.01 |
| 55 | —CH₂CH₂OH | >1000 | >1000 | 38 ± 2 | 0.81 ± 0.04 |
| 56 | —CH₂CH₂CH₂OH | 660 ± 20 | >1000 | 39 ± 1 | 2.1 ± 0.1 |
| 57 | —C(CH₃)₂OH | >1000 | >1000 | 180 ± 40 | 4.0 ± 0.3 |

Small molecule sialidase inhibitors may also include a compound having the following structural formula:

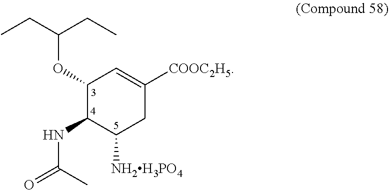

Compound 58, also known as oseltamivir ((1S,2S,3S,4R)-3-[(1S)-1-acetamido-2-ethyl-butyl]-4-(diaminomethylideneamino)-2-hydroxy-cyclopentane-1-carboxylic acid) is active in this carboxylate form as an inhibitor of all four human sialidases, but is a poor inhibitor as compared to the other small molecule sialidase inhibitors described above. Oseltamivir is a potent sialidase inhibitor in mice and therefore may be useful as a small molecule sialidase inhibitor in non-human patients in which it exhibits activity closer to its activity in mice.

The above compounds or other small molecule sialidase inhibitors may be administered in an amount and for a time sufficient to inhibit at least human one sialidase, prevent or control fibrocyte formation in a human, or prevent or inhibit fibrosis systemically in a human or in the area of administration in a human.

The dose may be sufficient to establish a systemic concentration or a concentration in the area of administration of at least 3 μM. Dosing may be daily for at least one week, at least two weeks, at least three weeks, or indefinitely.

The small molecule sialidase inhibitors may be administered via intravenous or intraperitoneal injection, orally, topically, or via inhalation.

Antibody Sialidase Inhibitors

Antibody sialidase inhibitors may include isolated human and humanized antibodies that bind to the active site of at least one human sialidase, with sufficient strength to inhibit the human sialidase activity or with a binding affinity of at least 10⁻⁷ M. The sialidase antigen may specifically include an arginine triad that binds the carboxylate group common to all sialic acids, a tyrosine/glutamic acid pair, an aspartic acid that acts as the acid/base catalyst, all as identified in Monti E., et al., Sialidases in vertebrates: a family of enzymes tailored for several cell functions. Adv Carbohydr Chem Biochem 64: 403-479 (2010), or any combinations of these antigens.

The antibody may be a monoclonal antibody. The antibody may further include antibody fragments able to inhibit human sialidase activity.

The antibody sialidase inhibitors may be administered in an amount and for a time sufficient to inhibit at least one human sialidase, prevent or control fibrocyte formation in a human, or prevent or inhibit fibrosis systemically in a human or in the area of administration in a human.

The antibody sialidase inhibitors may be administered via intravenous or intraperitoneal injection, topically, or via inhalation.

### Fibrosing Diseases

Sialidase inhibitors may be used to prevent or inhibit fibrosis in any of a number of fibrosing diseases in a mammal, particularly in a human.

For example, they may prevent or inhibit fibrosis occurring in the liver, kidney, lung, heart and pericardium, eye, skin, mouth, pancreas, gastrointestinal tract, brain, breast, bone marrow, bone and joints, genitourinary system, a tumor, including a cancerous tumor, or a wound.

Generally, they may prevent or inhibit fibrosis resulting from conditions including but not limited to rheumatoid arthritis, lupus, psoriatic arthritis, ankylosing spondylitis, pathogenic fibrosis, fibrosing disease, fibrotic lesions such as those formed after Schistosoma japonicum infection, radiation damage, autoimmune diseases, Lyme disease, chemotherapy induced fibrosis, HIV or infection-induced focal sclerosis, failed back syndrome due to spinal surgery scarring, abdominal adhesion post surgery scarring, fibrocystic formations, fibrosis after spinal injury, surgery-induced fibrosis, mucosal fibrosis, peritoneal fibrosis caused by dialysis, tumor-associated fibrosis, and Adalimumab-associated pulmonary fibrosis.

Specifically, in the liver, they may prevent or inhibit fibrosis resulting from conditions including but not limited to alcohol, drug, and/or chemically induced cirrhosis, ischemia-reperfusion injury after hepatic transplant, necrotizing hepatitis, hepatitis B, hepatitis C, primary biliary cirrhosis, primary sclerosing cholangitis, and steatosis.

Relating to the kidney, they may prevent or inhibit fibrosis resulting from conditions including but not limited to proliferative and sclerosing glomerulonephritis, nephrogenic fibrosing dermopathy, diabetic nephropathy, renal tubulointerstitial fibrosis, and focal segmental glomerulosclerosis.

Relating to the lung, they may prevent or inhibit fibrosis resulting from conditions including but not limited to pulmonary interstitial fibrosis, sarcoidosis, pulmonary fibrosis, idiopathic pulmonary fibrosis, asthma, chronic obstructive pulmonary disease, diffuse alveolar damage disease, pulmonary hypertension, neonatal bronchopulmonary dysplasia, chronic asthma, and emphysema. There are several sub-names or synonyms for pulmonary fibrosis including, but not limited to, cryptogenic fibrosing alveolitis, diffuse interstitial fibrosis, idiopathic interstitial pneumonitis, Hamman-Rich syndrome, silicosis, asbestosis, berylliosis, coal worker's pneumoconiosis, black lung disease, coal miner's disease, miner's asthma, anthracosis, and anthracosilicosis.

Relating to the heart and/or pericardium, they may prevent or inhibit fibrosis resulting from conditions including but not limited to myocardial fibrosis, atherosclerosis, coronary artery restenosis, congestive cardiomyopathy, dilated cardiomyopathy, heart failure, and other post-ischemic conditions.

Relating to the eye, they may prevent or inhibit fibrosis resulting from conditions including but not limited to exophthalmos of Grave's disease, proliferative vitreoretinopathy, anterior capsule cataract, corneal fibrosis, corneal scarring due to surgery, trabeculectomy-induced fibrosis, progressive subretinal fibrosis, multifocal granulomatous chorioretinitis, and other eye fibrosis.

Relating to the skin, they may prevent or inhibit fibrosis resulting from conditions including but not limited to Depuytren's contracture, scleroderma, keloid scarring, psoriasis, hypertrophic scarring due to burns, atherosclerosis, restenosis, and psuedoscleroderma caused by spinal cord injury.

Relating to the mouth and/or esophagus, they may prevent or inhibit fibrosis resulting from conditions including but not limited to periodontal disease scarring, gingival hypertrophy secondary to drugs, and congenital esophageal stenosis.

Relating to the pancreas, they may prevent or inhibit fibrosis resulting from conditions including but not limited to pancreatic fibrosis, stromal remodeling pancreatitis, and stromal fibrosis.

Relating to the gastrointestinal tract, they may prevent or inhibit fibrosis resulting from conditions including but not limited to collagenous colitis, villous atrophy, crypt hyperplasia, polyp formation, fibrosis of Crohn's disease, and healing gastric ulcer.

Relating to the brain, they may prevent or inhibit fibrosis resulting from conditions including but not limited to glial scar tissue.

Relating to the breast, they may prevent or inhibit fibrosis resulting from conditions including but not limited to fibrocystic disease and desmoplastic reaction to breast cancer.

Relating to the bone marrow, they may prevent or inhibit fibrosis resulting from conditions including but not limited to fibrosis in myelofibrosis, myelodysplasia and neoplastic diseases.

Relating to the bone, they may prevent or inhibit fibrosis resulting from conditions including but not limited to rheumatoid arthritis, systemic lupus erythematosus (SLE), psoriatic arthritis, ankylosing spondylitis, and rheumatoid pannus formation.

Relating to the genitourinary system, they may prevent or inhibit fibrosis resulting from conditions including but not limited to endometriosis, uterine fibroids, ovarian fibroids, and Peyronie's disease.

Relating to radiation-induced damage, they may prevent or inhibit fibrosis related to, but not limited to, treatment of head and neck cancer, ovarian cancer, prostate cancer, lung cancer, gastrointestinal cancer, colon cancer, and breast cancer.

### EXAMPLES

The following examples illustrate aspects of the invention; no example is intended to encompass the invention as a whole. Furthermore, although some examples may present discrete embodiments of the invention, aspects of such examples may be combined with other variations of the invention as described above or in different examples unless such combinations would be clearly inoperable to one of skill in the art.

Unless otherwise specified, for instance when discussing data derived from mouse samples, sialidases tested in these examples were human sialidases.

### Example 1

### Determining Sialidase Activity

In general, sialidase activity, including human sialidase activity, may be assessed at 37° C. in the presence of buffers ranging in pH from 3.7 to 7.4. Buffers with pH 3.7, 4.0, 4.6, 5.2 and 5.6 are 100 mM sodium acetate buffers. Buffers with pH 5.8, 6.4, 7.0 and 8.0 are based on Phosphate-Buffered Saline (PBS), with addition of 12N HCl or 1M NaOH to adjust the pH. Bovine serum albumin (BSA) is added to the buffer at a concentration of 100 ug/ml. Sialidase at a final concentration of 300 ng/ml is then added to the buffer. A sialidase inhibitor is then added to the mixture at a series of final concentrations. The reaction mixture is incubated for 30 minutes to allow the inhibitor to bind the sialidase. The fluorometric substrate 4MU-NANA [2′-(4-Methylumbelliferyl)-α-D-N-acetylneuraminic acid sodium salt hydrate is then added at a final concentration of 200 μM. Control reactions have no added inhibitor. The total volume of each reaction mixture is 0.1 ml. The cleavage of 4MU-NANA is then monitored by fluorescence every 20 minutes for 5 hours with excitation light at 360 nm and the fluorescence emission at 460 nm.

The fluorescence in the absence of sialidases is subtracted from all readings. The fluorescence of known concentrations of 4-methylumbelliferone is used to convert fluorescence to moles of product.

To determine if sialidases have enzymatic activity at the pH of the extracellular environment, sialidases may be assayed at pH 7.0, approximately corresponding to an extracellular pH in a normal tissue, at pH 6.4, approximately corresponding to an extracellular pH in a fibrotic tissue, or both.

In an example assay, recombinant human sialidases were assayed at pH 6.4, approximately corresponding to the extracellular pH that might occur in a fibrotic tissue, and at pH 7.0, approximately corresponding to a normal extracellular pH. Results are presented in Table 6. All four recombinant sialidases showed activity at pH 6.4 and pH 7.0, indicating that human sialidases can be active in an extracellular environment in both fibrotic and normal tissue.

TABLE 6
Sialidase Activity at pH 6.4 and pH 7
Activity, μmole/min/mg protein

| Sialidase | pH 6.4 | pH 7.0 |
|---|---|---|
| NEU1 | 12.8 ± 1.3 | 7.2 ± 0.9 |
| NEU2 | 16.4 ± 1.2 | 8.9 ± 1.2 |
| NEU3 | 15.2 ± 1.0 | 8.3 ± 1.2 |
| NEU4 | 3.8 ± 0.8 | 2.6 ± 0.9 |

### Example 2

### A Positive Feedback Pathway Involving NEU3 and IL-6 in Human PBMC

To determine the effects of NEU3 on IL-6 production in human PBMC, human peripheral blood was collected from healthy volunteers who gave written consent and with specific approval from the Texas A&M University human subjects review board. PBMC were isolated from the blood using Ficoll-Paque density gradient centrifugation (GE Healthcare, Cincinnati, Ohio) following the manufacture's protocol. PBMCs were cultured at 10⁵ cells/ml in each well of 96-well flat bottom tissue culture plates (VWR, Radnor, Pa.) with RPMI-1640 (VWR) supplemented with 10% bovine calf serum (BCS) (VWR), 100 U/ml penicillin, 100 μg/ml streptomycin, (VWR) and 2 mM glutamine (VWR) in a final volume of 200 μl per well. Cells were also cultured at 10⁵ cells/ml in serum-free medium as described previously in Pilling, D., Vakil, V. & Gomer, R. H. Improved serum-free culture conditions for the differentiation of human and murine fibrocytes. Journal of immunological methods 351, 62-70, (2009) (incorporated by reference herein) in RPMI-1640 supplemented with 10 mM HEPES (VWR), 1× non-essential amino acids (VWR), 1 mM sodium pyruvate (VWR), 2 mM glutamine (VWR), 100 U/ml penicillin, 100 μg/ml streptomycin (VWR), and 1× ITS-3 (Sigma-Aldrich, St. Louis, Mo.) in a final volume of 200 μl per well. When the cells were plated, recombinant human sialidase NEU3 (TP316537, Origene, Rockville, Md.), was added to a final concentration of 0-500 ng/ml. The NEU3 was diluted in RPMI-1640 medium with or without serum and added to cells to make the total volume in a well 200 μl. The cells were then incubated at 37° C. with 5% CO₂.

The culture supernatants were collected after two or five days and assayed using an IL-6 ELISA kit (BioLegend, San Diego, Calif.) following the manufacturer's protocol, reading absorbance with a SynergyMX plate reader (BioTek, Winooski, Vt.). Statistics were analyzed using Prism software (Graphpad, La Jolla, Calif.).

Results are presented in FIGS. 1B-1E. In the presence or absence of serum, NEU3 significantly upregulated the extracellular accumulation of IL-6 by human immune cells. The accumulated levels of IL-6 were comparable to or higher than the normal human serum levels of IL-6, which are 0-20 pg/ml.

To determine the effects of IL-6 on NEU3 production by human PBMC, PBMC were cultured as above in serum-free medium at 10⁵ cells/ml with 2 ml/well in 6-well tissue culture plates (VWR) in the presence or absence of recombinant human interleukin-6 (IL-6) (BioLegend). After three days, the medium was carefully removed and the cells were washed with 1 ml of phosphate buffered saline (PBS) at room temperature. The cells were detached with 500 μl of Accutase cell detachment solution (VWR) per well for 6 minutes at 37° C. 1000 μl RPMI medium supplemented as described above with BCS, penicillin, streptomycin and glutamine was added per well to neutralize the Accutase. After pipetting the cell solutions 4 times, the cells were placed in sterile 1.7 ml microtubes (Genesee Scientific, San Diego, Calif.) and cells were collected by centrifugation at 5000×g for 10 minutes at 4° C. The pelleted cells were washed twice by resuspension with 1000 μl of ice-cold PBS and centrifugation. The cells were resuspended in 200 μl of ice-cold 2% (w/v) paraformaldehyde (EMS, Hatfield, Pa.), in PBS for 10 minutes on ice for fixation. 1000 μl of ice-cold PBS was added and cells were collected by centrifugation. The cells were resuspended in 200 μl of ice-cold 2% (w/v) bovine serum albumin (BSA) (VWR) in PBS (PBSA), for 10 minutes on ice for blocking. 1000 μl of ice-cold PBS was added and cells were collected by centrifugation. The pellet was resuspended in 200 μl of ice-cold 0.1% (w/v) Triton X-100 (Alfa Aesar, Ward Hill, Mass.) in PBS and membranes were lysed for 10 minutes on ice. 1000 μl of ice-cold PBS was added and cells were collected by centrifugation. Cells were resuspended in 500 μl of PBSA, and 125 μl was then collected by centrifugation for a staining reaction.

The pelleted cells for a staining reaction were resuspended in 100 μl of 1 μg/ml rabbit polyclonal anti-NEU1 (TA335236, Origene), anti-NEU2, (TA324482, Origene,) anti-NEU4 (AP52856PU-N, Acris/Origene), irrelevant rabbit polyclonal antibody (AB-105-C, R&D Systems, Minneapolis, Minn.), or no antibody in 2% (w/v) PBSA, or 1 μg/ml anti-NEU3 (TA590228, Origene) in 2% (w/v) PBSA with 0.1% (v/v) NP-40 alternative (EMD Millipore, Billerica, Mass.). Cells were incubated with antibodies for 60 minutes on ice. 500 μl of ice-cold PBS was added and cells were collected by centrifugation. Cells were washed twice by resuspension in 1000 μl of ice-cold PBS centrifugation. The cells were then incubated with 100 μl of 1:1000 goat anti-rabbit Alexa Fluor 647 (Life Technologies, Carlsbad, Calif.), in PBSA for 30 minutes on ice. The cells were then washed twice as described above. The cells were then resuspended in 100 μl of in PBSA, kept on ice, the fluorescence of cells was analyzed on an Accuri C6 flow cytometer (BD Bioscience), using forward- and side-scatter to identify monocytes and lymphocytes as described previously in Cox, N., Pilling, D. and Gomer, R. H. DC-SIGN activation mediates the differential effects of SAP and CRP on the innate immune system and inhibits fibrosis in mice. Proceedings of the National Academy of Sciences of the United States of America 112, 8385-8390 (2015) (incorporated by reference herein).

Results are presented in FIG. 1F and FIG. 1G. The data shows that 100 and 1000 pg/ml of IL-6 significantly increased levels of the sialidase NEU3 in human monocytes and lymphocytes.

Overall, the data in FIGS. 1B-1F demonstrates the ability of the recombinant human sialidase NEU3 to increase levels of the pro-fibrotic cytokine IL-6 in human immune system cells, and the ability of recombinant human IL-6 to increase NEU3 levels in human immune system cells, supporting the existence of a positive feedback loop of the type depicted in FIG. 1A in which NEU3 contributes to fibrosis. This suggests that sialidase inhibitors are a suitable therapeutic for fibrosing diseases by inhibiting the NEU3 component of the feedback loop to decrease IL-6 levels and thus inhibit fibrosis.

### Example 3

### Sialidases In Human Lungs

Glass slides with serial 5 μm thick HEPES-Glutamic acid buffer mediated Organic solvent Protection Effect (HOPE)-fixed sections from patients with chronic obstructive pulmonary disease (COPD) or Interstitial Lung Disease (ILD), a type of pulmonary fibrosis, were obtained from the National Heart Lung and Blood Institute-sponsored Lung Tissue Research Consortium (LTRC), with specific approval of the Texas A&M Institutional Board. Written consent was received and all samples were de-identified before analysis. ILD <50% forced expiratory volume in 1 second (FEV1) indicates a pulmonary fibrosis patient with poor lung function. FEV1 is the volume exhaled during the first second of a forced expiratory maneuver started from the level of total lung capacity.

The slides were treated with 60° C. isopropanol for 10 minutes, then treated with fresh 60° C. isopropanol for a further 15 minutes. Slides were rehydrated in 70% acetone (v/v in distilled water) for 20 minutes, and then distilled water for 5 minutes.

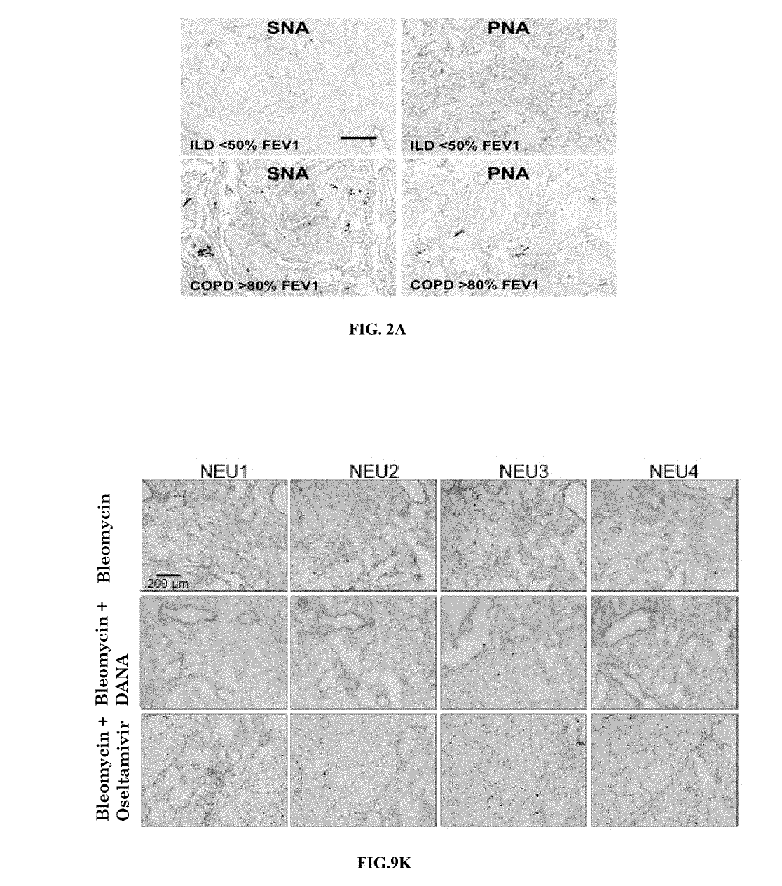

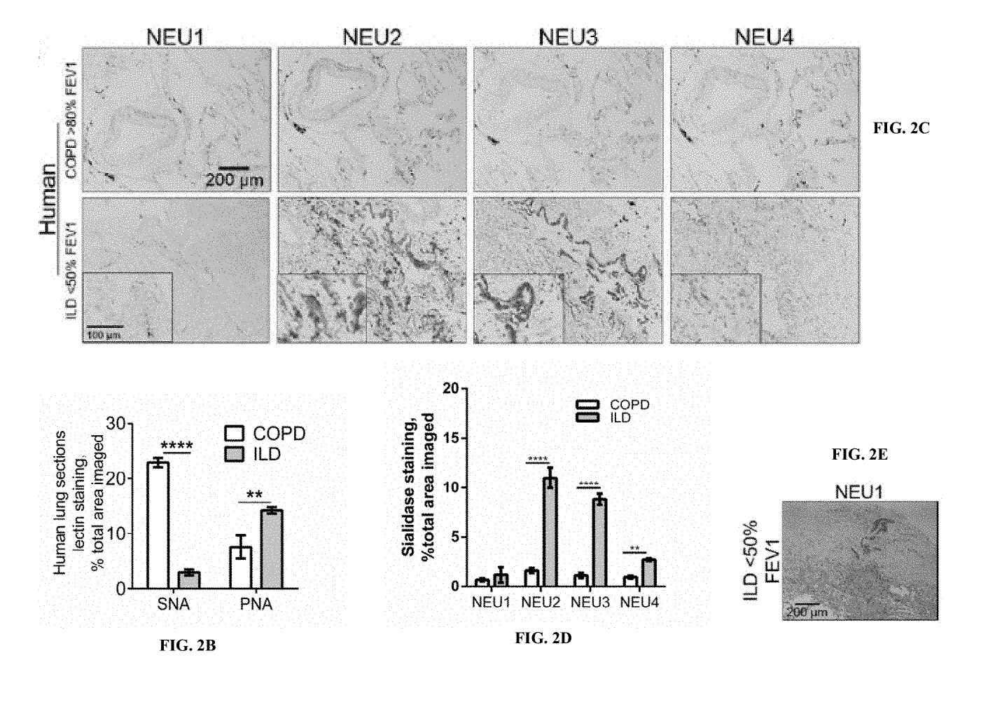

Certain slides were then stained with 2 μg/ml of biotinylated Sambucus Nigra agglutinin (SNA) lectin or 2 μg/ml of biotinylated Peanut agglutinin (PNA) lectin (both from Vector Laboratories) in PBS-BSA following the manufacturer's directions. The staining was revealed using ExtrAvidin-Alkaline phosphatase and Vector Red Alkaline Phosphatase following the manufacturer's directions and slides were then counterstained with hematoxylin. Representative results of staining are presented in FIG. 2A. Staining results quantified using ImageJ are presented in FIG. 2B. Lung tissue from patients with pulmonary fibrosis contained fewer polysaccharides with sialic acid having an α(2,6)-linkage, as shown by SNA staining, than lung tissue from COPD patients, despite having more carbohydrates overall, as shown by PNA staining, than COPD patients. This indicates an increase in sialidase activity in patients with pulmonary fibrosis as compared to patients without fibrosis, even when those patients had another lung disease.

Other slides were blocked and stained with antibodies. Non-specific binding was blocked with 2% nuclease-free/protease-free bovine serum albumin (VWR) in phosphate-buffered saline (PBS-BSA), for 30 minutes and the slides were then incubated at 4° C. overnight with rabbit polyclonal anti-NEU1 antibody (TA335236, Origene, Rockville, Md.), anti-NEU2 antibody (TA324482, Origene), or anti-NEU4 antibody (AP52856PU-N, Acris/Origene) at 1 μg/ml in PBS-BSA, or anti-NEU3 antibody (TA590228, Origene) at 1 μg/ml in PBS-BSA.

Slides were then washed in three changes of PBS over 30 minutes and incubated for 30 minutes with 2 microgram/ml biotinylated donkey anti-rabbit IgG (711-066-152, Jackson ImmunoResearch Laboratories, Inc., West Grove, Pa.) in PBS. After washing, the biotinylated antibodies were detected by streptavidin-alkaline phosphatase (Vector Laboratories, Burlingame, Calif.) following the manufacturer's protocol. After mounting the slides with VectaMount (Vector Laboratories), images were captured using a Nikon Microphot-FX microscope (Nikon, Melville, N.Y.) with an A35100U CCD camera (OMAX, Gyeonggi-do, South Korea) and ToupView (Touptek Photonics, Hangzhou, China). Representative results are presented in FIG. 2C. Staining results quantified using ImageJ are presented in FIG. 2D.

It is clear from these results that NEU2, NEU3, and NEU4 are expressed at increased levels in patients with pulmonary fibrosis as compared to patients without fibrosis, even when those patients had another lung disease (COPD, which showed low levels of all four sialidases). Results were less clear for NEU1, with two of three patients with fibrosis showing only low levels of NEU1, but with the third patient showing increased levels (FIG. 2E). Higher magnification images showed patchy distributions of the upregulated sialidases in fibrotic lesions. These results also lead one to expect increased sialidase activity in patients with fibrosis, particularly for the sialidases NEU2, NEU3, and NEU4.

### Example 4

### Sialidases In Cardiac Fibrosis

A piece of a normal human heart and a piece of a fibrotic region of a heart from a patient with dilated cardiomyopathy were fixed in formalin, embedded in paraffin, and sectioned. The sections were a gift from Dr. JoAnn Trial at Baylor College of Medicine, Houston, Tex. The slides with heart sections were incubated for 10 minutes in xylene to remove paraffin. Then the sections were hydrated by immersing them sequentially in 100%, 95%, 70% ethanol (in water) and then water for 5-10 minutes at each step. The sections were washed twice for 5 minutes in PBS. The sections were then immersed in 10 mM sodium citrate/0.05% Tween-20, pH 6.0, preheated to 97-98° C. and treated at this temperature for 20 minutes. Subsequent steps were at room temperature unless noted otherwise.

The sections were incubated for 5 minutes in water and then 5 minutes in PBS. The sections were incubated with PBS containing 2% bovine serum albumin (VWR) (PBSA) for 30 minutes. The PBSA was removed and then 1-2 drops Avidin blocking reagent (SK-2002, Vector Laboratories) was added for 10-15 minutes. The sections were rinsed in 50 ml of PBS, twice for 5 minutes. The sections were incubated in 1-2 drops of biotin blocking reagent (SK-2002, Vector Laboratories) for 10-15 minutes. The sections were rinsed in 50 ml of PBS, twice for 5 minutes. The sections were incubated in 1 μg/ml rabbit polyclonal anti-NEU3 antibody (TA590228, Origene) diluted in 2% PBSA with 0.01% NP-40 alternative (EMD Millipore) and 0.01% sodium dodecyl sulphate (VWR), overnight at 4° C. The sections were washed twice in 50 ml of PBS for 10 minutes. The sections were then incubated with 1:5000 biotin labelled donkey-anti-rabbit secondary antibody (Jackson, West Grove, Pa.) in 2% PBSA for 30 minutes. The sections were rinsed in 50 ml of PBS, twice for 5 minutes. The sections were incubated with 1:500 ExtrAvidin-Alkaline Phosphatase (Vector) in 2% PBSA for 30 minutes. The sections were washed twice in 50 ml of PBS, for 10 minutes each. The sections were washed once in 50 ml water, and then incubated for 5 minutes in 50 ml of 100 mM Tris/HCl pH 8.2. The sections were incubated for 5-10 minutes with Vector Red Alkaline Phosphatase reagent (Vector) following the manufacture's protocol. After 7 minutes, the sections were washed in 100 mM Tris/HCl pH 8.2 for 5 minutes. The sections were rinsed in water, and counterstained with Gill's #3 hematoxylin for 10 seconds. The sections were washed in water once for 5 minutes. Then the slides were incubated for 30 seconds in Scott's Tap Water. The sections were dehydrated by immersing them sequentially in 70%, 95% and 100% ethanol, and then xylene for 5 minutes at each step. The sections were mounted with permanent mounting medium (EMS, Hatfield, Pa.) under a coverslip.

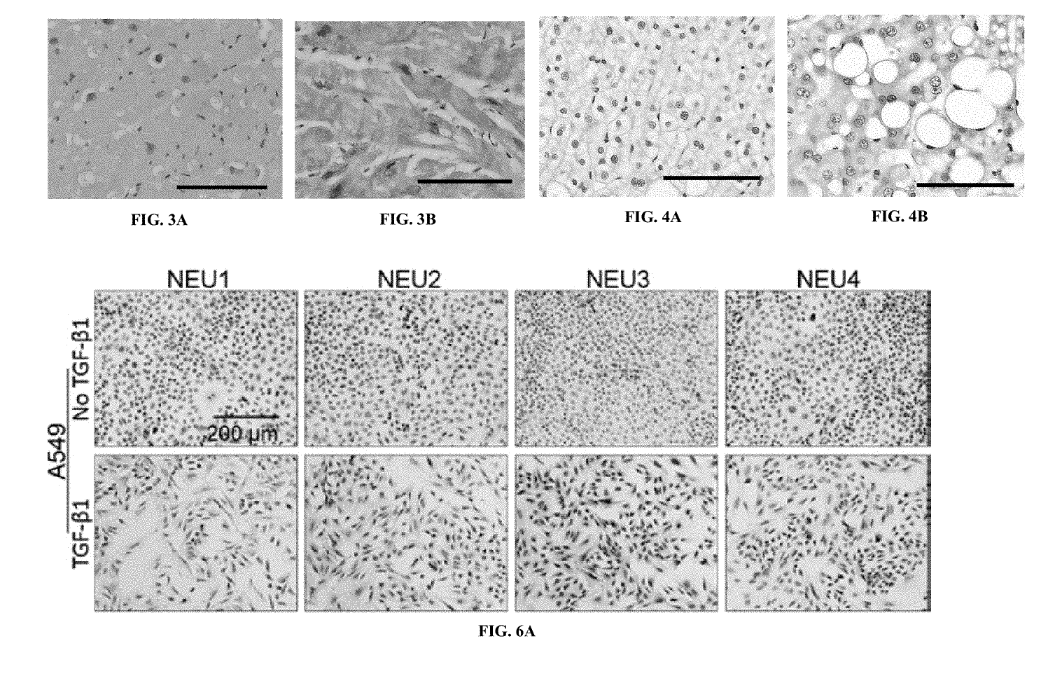

Positive staining for NEU3 was red. Compared to a normal human heart (FIG. 3A), the fibrotic heart (FIG. 3B) showed intense staining for NEU3. This demonstrates increased expression of NEU3 in human cardiac fibrosis, consistent with results obtained for other human fibroses.

### Example 5

### Sialidases In Liver Fibrosis

A piece of a normal human liver and a piece of liver from a patient with steatosis and fibrosis of the liver were fixed in formalin, embedded in paraffin, and sectioned. The sections were a gift from Dr. Dr. Tatiana Kisseleva at the University of California at San Diego. Subsequent staining was performed as described for the normal and fibrotic human heart sections on Example 4.

Positive staining for NEU3 was red. Compared to a normal human liver (FIG. 4A), the liver with steatosis and fibrosis (FIG. 4B) showed intense staining for NEU3. This demonstrates increased expression of NEU3 in human liver fibrosis, consistent with results obtained for other human fibroses.

### Example 6

### Effects of Sialidases on Fibrocyte Formation in Humans and SAP Inhibition of Fibrocyte Formation

Human peripheral blood was collected from healthy adult volunteers who gave written consent and with specific approval from the Texas A&M University human subjects Institutional Review Board. Peripheral blood mononuclear cells (PBMC) were isolated from blood using Ficoll-Paque Plus (GE Healthcare Biosciences, Piscataway, N.J.). Human PBMC were incubated in serum-free medium (FibroLife basal media (LM-0001, Lifeline Cell Technology, Walkersville, Md.), supplemented with 10 mM HEPES (Sigma-Aldrich, St. Louis, Mo.), 1× non-essential amino acids (Sigma-Aldrich), 1 mM sodium pyruvate (Sigma-Aldrich), 2 mM glutamine (Invitrogen, Carlsbad, Calif.), 100 U/ml penicillin, 100 μg/ml streptomycin (Sigma-Aldrich), and 1× ITS-3 (Sigma-Aldrich) in the presence of absence of 100 ng/ml recombinant human sialidases (Origene, Rockville Md.), 10 μg/ml DANA (Calbiochem, San Diego, Calif.), or 1 μg/ml human SAP (Calbiochem). After 5 days, fibrocytes were counted, and results are presented in FIG. 5.

NEU2 and NEU4, when added to cultures of human PBMC, potentiated fibrocyte formation. DANA inhibited fibrocyte differentiation, and DANA inhibited the NEU4 potentiation of fibrocyte differentiation, indicating that decreasing sialidase activity can decrease fibrocyte formation.

NEU2, NEU3, and NEU4 partially counteracted the ability of SAP to inhibit fibrocyte formation, indicating that desialyation of SAP may be at least partially responsible for the potentiation of fibrocyte formation by sialidases.

NEU1, however, did not potentiate fibrocyte differentiation or counteract SAP in this assay.

### Example 7

### TGF-β1 Increases Sialidase Expression in Human Cells

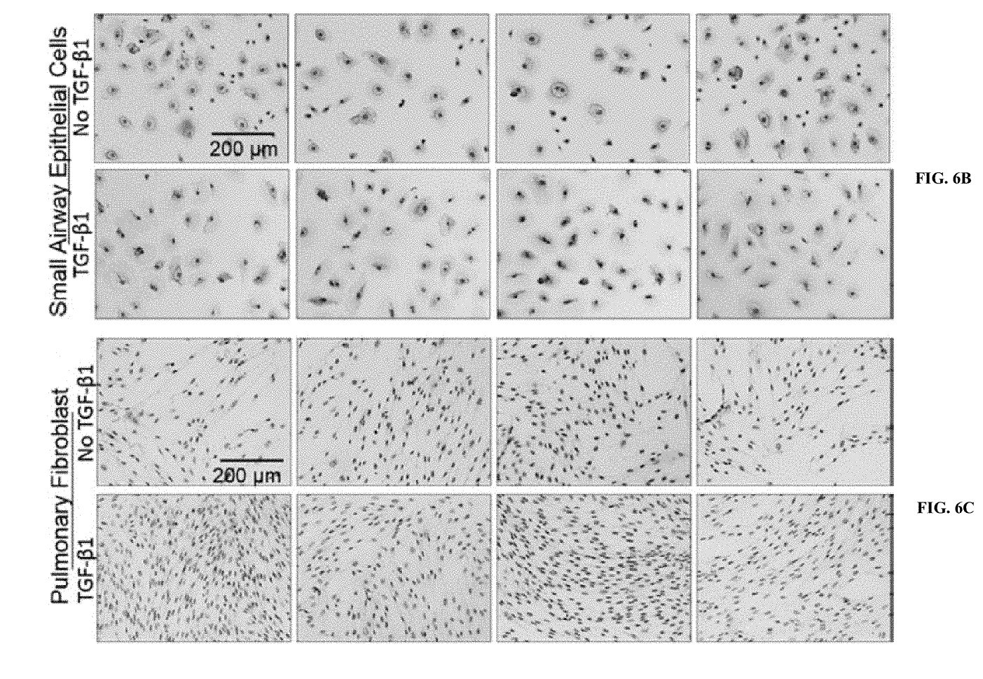

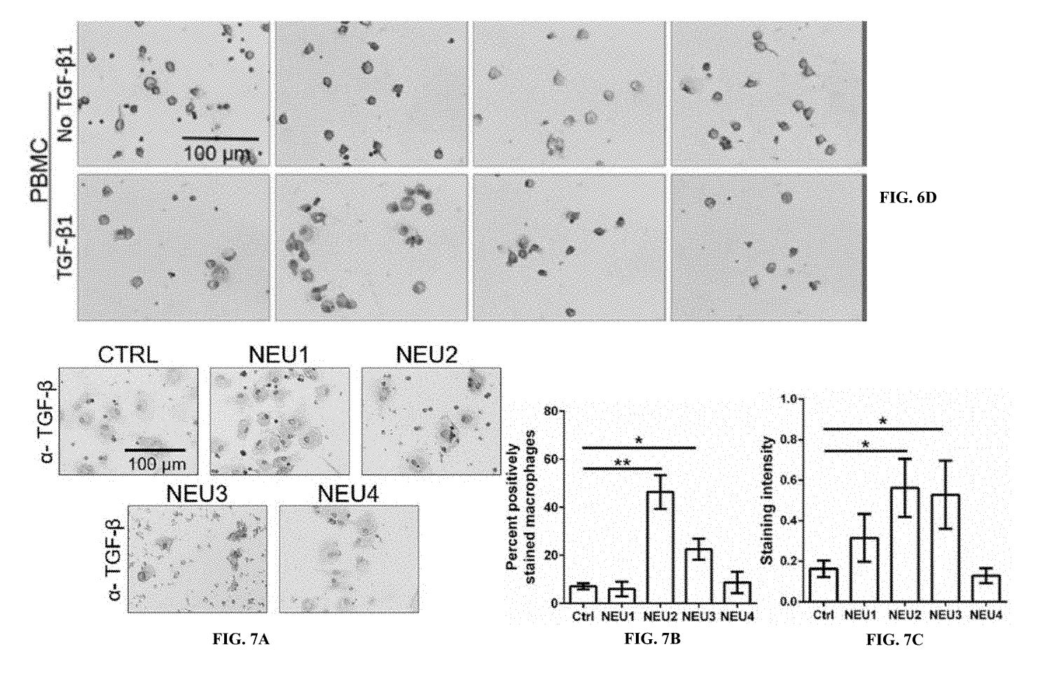

TGF-β1 is strongly associated with fibrosis so its effects on sialidase expression were determined. Human lung adenocarcinoma cell line A549, human small airway epithelial cells, human pulmonary fibroblasts, and human immune cells were cultured with or without 10 ng/ml of recombinant active TGF-β1, which is a standard concentration used in tissue culture experiments. After three days for A549 cells, airway epithelial cells, and fibroblasts, or five days for immune cells, the cells were stained with antibodies against sialidases. Results are presented in FIGS. 6A-4D.

TGF-β1 caused A549 cells to undergo a characteristic change in morphology and to increase levels of NEU3 (FIG. 6A). TGF-β1 also caused human small airway epithelial cells to increase levels of NEU3, and slightly increased levels of NEU1 (FIG. 6B). TGF-β1 increased the proliferation of human pulmonary fibroblasts, and caused these cells to increase levels of NEU3 (FIG. 6C). TGF-β1 increased levels of NEU2 and NEU3 in some cells in cultures of human PBMC (FIG. 6D). Results in each figure are representative of three independent experiments.

Thus, sialidase expression and particularly NEU3 expression, is increased by TGF-β1.

### Example 8

### NEU2 and NEU3 Upregulate the Intracellular and Extracellular Accumulation of TGF-β1 by PBMC

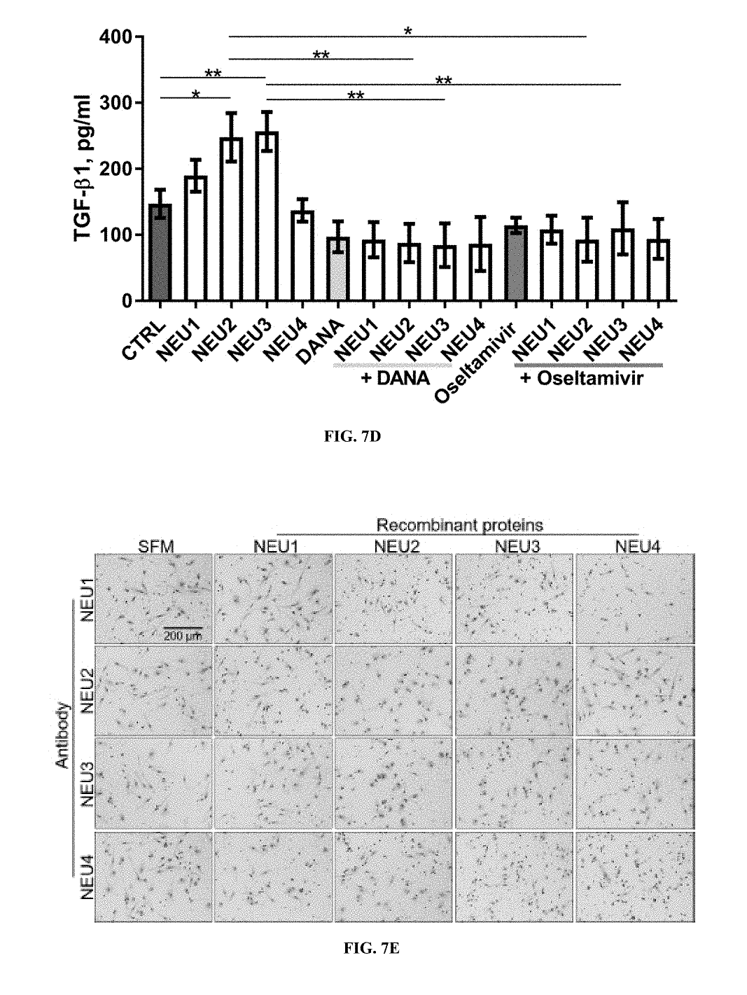

To determine if sialidases might cause cells to accumulate intracellular and extracellular TGF-β1, human PBMC were cultured with sialidases in 96 well plates at 5×10⁵ cells/ml and 200 μl/well in FibroLife serum-free medium for five days, then air dried. The levels of TGF-β1 in or on the cells was detected by staining with an antibody against TGF-β1. Photomicrographs representative of five independent samples are presented in FIG. 7A. Photomicrographs were analyzed by counting stained cells (FIG. 7B) and quantifying staining intensity using ImageJ (FIG. 7C).

PBMC were cultured in 96 well plates at 5×10⁵ cells/ml and 200 μl/well in FibroLife serum-free medium. When the cells were plated, recombinant human sialidases were added to final concentrations of 200 ng/ml, and DANA or oseltamivir were added to 3 μM. After 5 days, culture supernatants were analyzed using a TGF-β1 ELISA kit (R&D Systems). Results are presented in FIG. 7D.

When added to human PBMC, human NEU1 and NEU4 had no significant effect on the accumulation of TGF-β1, while NEU2 and NEU3 increased both cell-associated and extracellular TGF-β1. The addition of the sialidase inhibitors DANA or oseltamivir blocked the effects of NEU2 and NEU3 on extracellular TGF-β1, indicating that the effects of NEU2 and NEU3 are due to their sialidase activities. These data suggest that sialidases might be able to potentiate fibrosis by increasing levels of extracellular TGF-β1.

In a similar experiment, cells were stained for NEU1, NEU2, NEU3, or NEU4 using antibodies. Images representative of three experiments are provided in FIG. 7E. NEU 2 also caused some cells in PBMC to upregulate levels of NEU3. This indicates that one sialidase may increase expression of another sialidase, also consistent with a positive feedback loop.

### Example 9

### Sialidases In Mouse Lungs

4 to 6 week-old 20 g C57BL/6 mice (Jackson, Bar Harbor, Me.) were treated with an oropharyngeal aspiration of 50 μL of 0.9% saline, or 3 U/kg bleomycin (Calbiochem, EMD Millipore, Billerica, Mass.) in 50 μL of 0.9% saline, as described previously by Lakatos, H F. et al., Oropharyngeal aspiration of a silica suspension produces a superior model of silicosis in the mouse when compared to intratracheal instillation, Experimental Lung Research 32:181-199 (2006). The successful aspiration of bleomycin into the lungs was confirmed by listening for a crackling noise heard after the aspiration. Mice were weighed daily, and euthanized using CO₂ following NIH guidelines at day 21 after bleomycin aspiration. The experiment was performed in accordance with the recommendations in the Guide for the Care and Use of Laboratory Animals of the National Institutes of Health. The Texas A&M University Animal Use and Care Committee approved the protocol.

After euthanasia, the lungs were perfused with 300 microliters of phosphate buffered saline (PBS), pH 7.4, to collect bronchoalveolar lavage fluid (BAL) as described previously by Lakatos, H F. et al., Oropharyngeal aspiration of a silica suspension produces a superior model of silicosis in the mouse when compared to intratracheal instillation, Experimental Lung Research 32:181-199 (2006). The BALs were clarified by centrifugation at 500×g for 10 minutes, and the supernatants were transferred to Eppendorf tubes. The supernatants were flash frozen with liquid nitrogen, and stored at −20° C. until further use.

After collection of BAL fluid, mouse lungs were inflated with prewarmed Optimal Cutting Temperature (OCT) embedding medium (VWR, Radnor, Pa.) and then embedded in OCT, frozen on dry ice, and then stored at −80° C. 6-micron cryosections of mouse lungs were mounted on Superfrost Plus® microscope slides (VWR). The sections on the slides were allowed to air-dry for 24-48 hours. The slides were then fixed in acetone at room temperature for 20 minutes and air-dried for 10 minutes.

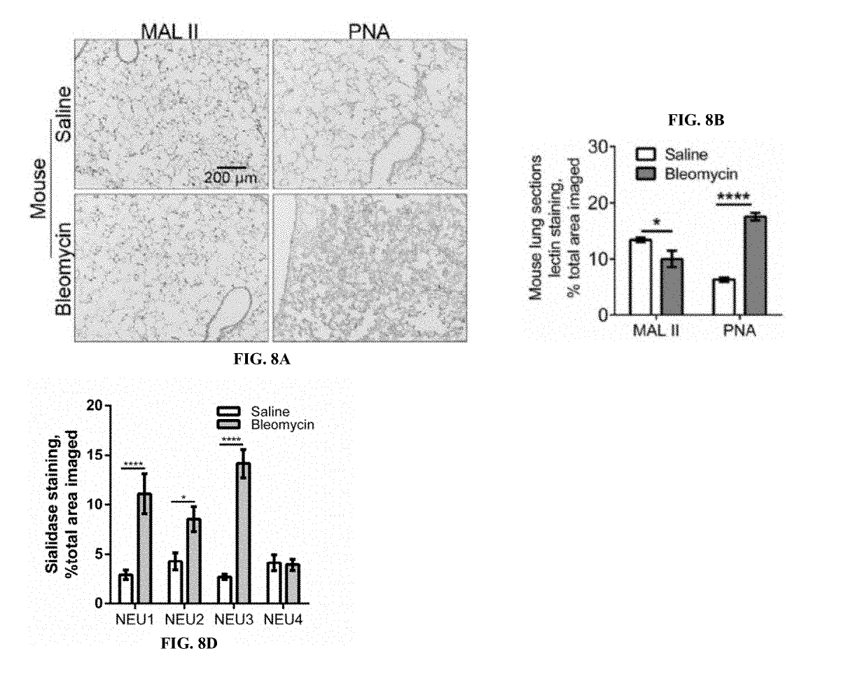

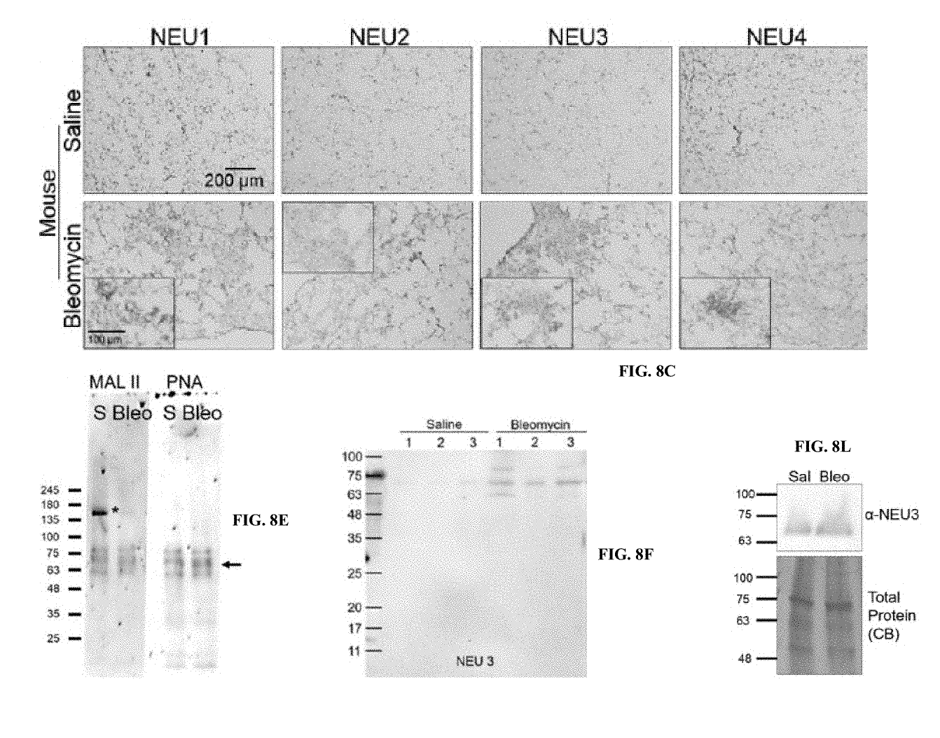

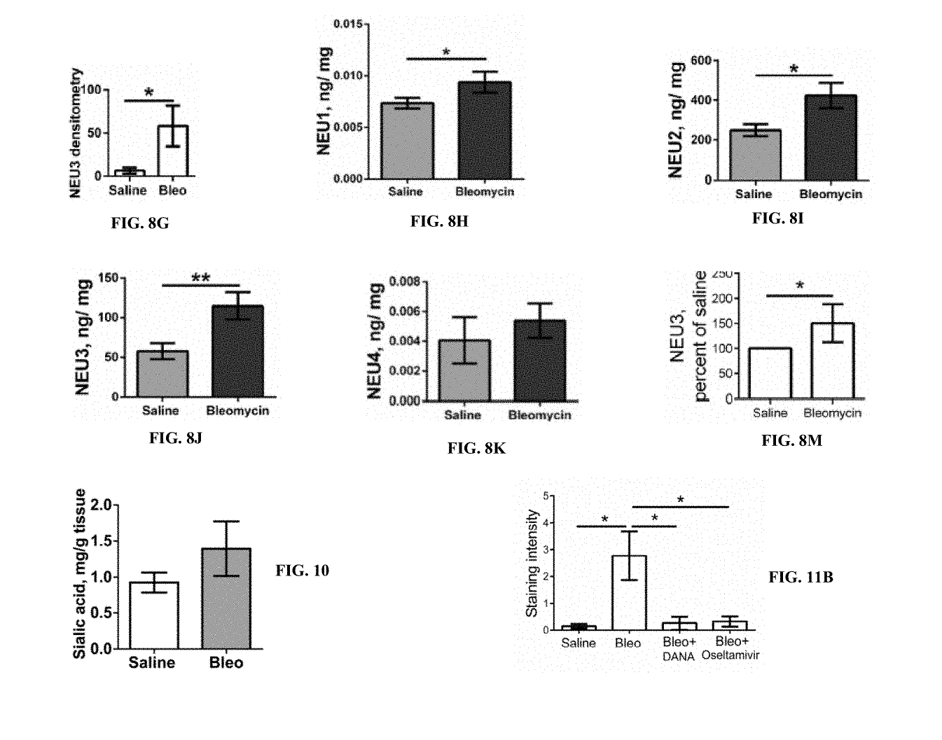

Some slides were then stained with 2 μg/ml of biotinylated Maackia amurensis lectin II (MAL II) (Vector Labs), or PNA, as described above. Representative results of staining are presented in FIG. 8A. Staining results quantified using ImageJ are presented in FIG. 8B. Lung tissue from mice with bleomycin-induced fibrosis contained fewer polysaccharides with sialic acid having an α(2,3)-linkage, as shown by MAL II staining, despite having more carbohydrates overall, as shown by PNA staining, than lung tissue from mice with no fibrosis. This indicates an increase in sialidase activity in fibrosis and, combined with the human lung data of FIGS. 2A-2E, indicates that the decrease in sialidase activity is not specific to a particular linkage.

Slides were also blocked and stained generally as described above in Example 3, except that slides were then incubated at 4° C. overnight with anti-NEU1 antibody, anti-NEU2 antibody, or anti-NEU4 at 1 μg/ml in PBS-BSA, or anti-NEU3 at 0.5 μg/ml in PBS-BSA containing an additional 0.5 M NaCl and 0.1% NP-40 Alternative (EMD Millipore, Billerica, Mass.) (PBSSN).

Representative results are presented in FIG. 8C. Staining results quantified using ImageJ are presented in FIG. 8D. It is clear from these results that NEU1, NEU2, and NEU3 are expressed at increased levels in mice with bleomycin-induced fibrosis as compared to mice with no fibrosis. No increase in NEU4 was observed. Higher magnification images showed patchy distributions of the upregulated sialidases in fibrotic lesions. These results also lead one to expect increased sialidase activity during fibrosis, particularly with respect to NEU1, NEU2, and NEU3.

Western blot analysis was also performed on the mouse BAL. 20 μl of BAL supernatants were mixed with Laemmli's sample buffer containing 20 mM dithiothreitol and heated to 100° C. for 5 minutes. Samples were loaded onto 4-20% Tris/glycine polyacrylamide gels (Lonza, Rockland, Me.).

Proteins were transferred to polyvinylidene difluoride (Immobilon P; Millipore, Bedford, Mass.) membranes in Tris/glycine/SDS buffer containing 20% methanol following the manufacturer's protocol. Membranes were blocked overnight at 4° C. with PBS containing 1% BSA and 5% nonfat milk protein and were then incubated overnight at 4° C. with 2 μg/ml of either biotinylated MAL II or biotinylated PNA. Membranes were then washed with PBS containing 0.1% Tween-20 over 30 minutes and were then incubated for 1 hour at room temperature with horseradish peroxidase-Streptavidin (BioLegend, San Diego, Calif.) diluted 1:5,000 in PBS-BSA. SuperSignal West Pico Chemiluminescence Substrate (Thermo Scientific, Rockford, Ill.) was used following the manufacturer's protocol to visualize the peroxidase, using a ChemiDoc XRS+ System (Bio-Rad, Hercules, Calif.). Results are shown in FIG. 8E. * indicates where a protein exhibited decreased sialyation in mice with bleomycin-induced fibrosis. The arrow indicates where a desialylated protein is present in these mice. These results are consistent with increased sialidase activity during fibrosis.

BAL proteins were also transferred to polyvinylidene difluoride (Immobilon P; Millipore, Bedford, Mass.) membranes in Tris/glycine/SDS buffer containing 20% methanol following the manufacturer's protocol. Membranes were blocked overnight at 4° C. with PBS containing 1% BSA and 5% nonfat milk protein and were then incubated overnight at 4° C. with anti-neuraminidase 3 (TA590228, Origene) at 0.18 microgram/ml in PBS-BSA. Membranes were then washed with PBS containing 0.1% Tween-20 over 30 minutes and were incubated with biotinylated donkey anti-rabbit (0.2 micrograms/ml) (711-066-152 Jackson ImmunoResearch Laboratories, West Grove, Pa.) in PBS-BSA for 1 hour at room temperature. Blots were washed in PBS containing 0.1% Tween-20 and further incubated for 1 hour at room temperature with horseradish peroxidase-Streptavidin (BioLegend, San Diego, Calif.) diluted 1:5,000 in PBS-BSA. SuperSignal West Pico Chemiluminescence Substrate (Thermo Scientific, Rockford, Ill.) was used following the manufacturer's protocol to visualize the peroxidase, using a ChemiDoc XRS+ System (Bio-Rad, Hercules, Calif.).

Results are presented in FIG. 8F and quantified in FIG. 8G. Increased amounts of NEU3 are clearly shown in the BAL from mice with bleomycin-induced fibrosis, as opposed to BAL from mice without fibrosis, while NEU1, NEU2, and NEU4 are not detected (data now shown). This is also consistent with increased sialidase levels, particularly of NEU3, during fibrosis.

Lysates from the indicated treatment groups were assayed for total protein and assayed by ELISA for NEU1 (FIG. 8H), NEU2 (FIG. 81), NEU3 (FIG. 8J), or NEU4 (FIG. 8K). In particular, lung tissue lysates were diluted to 100 μg total protein/ml in PBS. 55 μl of diluted lysate was added to a well of a 96-well Maxisorp immuno plate (Thermo Scientific) and incubated at 4° C. overnight. Serial dilutions of recombinant NEU 1, 2, 3, and 4 (Origene) in PBS were also incubated and used for standard curves. The solutions were removed and the wells were blocked with 200 μl PBS-BSA for 2 hours at room temperature with shaking. Anti-NEU1, 2, 3 and 4 antibodies (Origene) were then added in PBS-BSA for 1 hour at room temperature following the manufacturer's directions. After washing with PBS-BSA, 1:1000 HRP-conjugated donkey-anti-rabbit IgG (Jackson) in PBS-BSA was added for 1 hour. After washing, bound antibodies were detected using a TMB color development kit (BioLegend) and the reaction was stopped with 1 N HCl. Absorbances at 450 nm and 550 nm were measured using a SynergyMX plate reader.

Western blots of lung tissue lysate were also prepared and assayed for NEU3 using an anti-NEU3 antibody and a Coomassie brilliant blue stain for total protein (FIG. 8L). NEU3 and total protein were quantified (FIG. 8M).

Compared to saline controls, the bleomycin-treated mice had significantly higher levels of NEU1, NEU2 and NEU3, but not NEU4, in their lung lysates. In addition, on Western blots, compared to saline, bleomycin-treated mouse lung tissue lysates had significantly upregulated levels of NEU3. These results are also consistent with increased sialidase levels, particularly of NEU3, during fibrosis.

### Example 10

### Sialidase Inhibitors Decrease Fibrosis in Mice and Decrease Sialidase Levels

DANA inhibits all mammalian sialidases. Although oseltamivir is a poor inhibitor of human sialidases, it is a potent inhibitor of murine sialidases, and inhibits LPS-induced mouse macrophage sialidase activity with an IC₅₀ of 1 μm.

To demonstrate that sialidase inhibitors decrease fibrosis, mice were treated with oropharyngeal bleomycin to induce symptoms of pulmonary fibrosis as described for Example 9. Then, starting 24 hours after bleomycin had been administered, the mice were given daily intraperitoneal injections of 10 mg/kg DANA (Calbiochem) in PBS or 10 mg/kg oseltamivir (Sigma) in PBS. Both inhibitors are quite polar and thus probably remain in the extracellular space. Assuming 2 ml of extracellular space in a 20 gram mouse, these doses would then be ˜3 μM. At day 21, mice were sacrificed and BAL and cryosections of lungs were obtained as described for Example 3. The experiment was performed in accordance with the recommendations in the Guide for the Care and Use of Laboratory Animals of the National Institutes of Health. The Texas A&M University Animal Use and Care Committee approved the protocol.

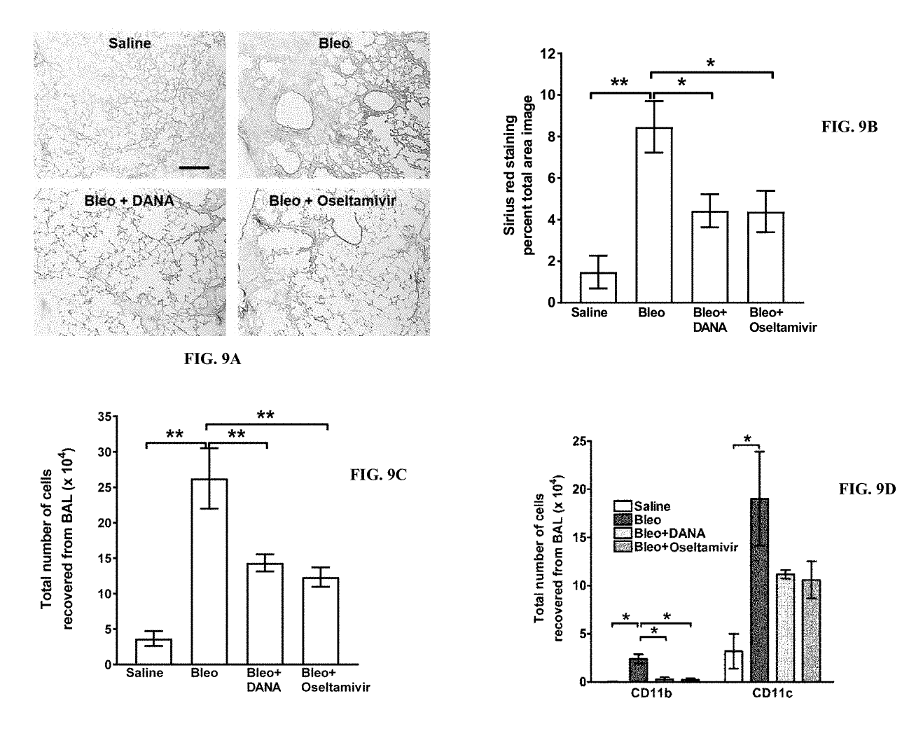

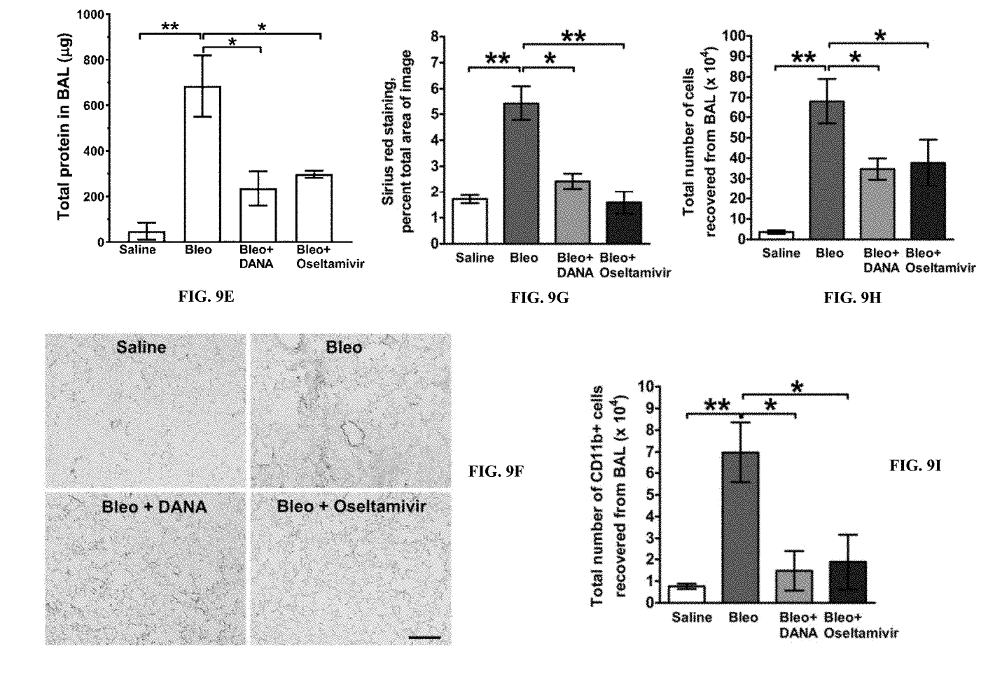

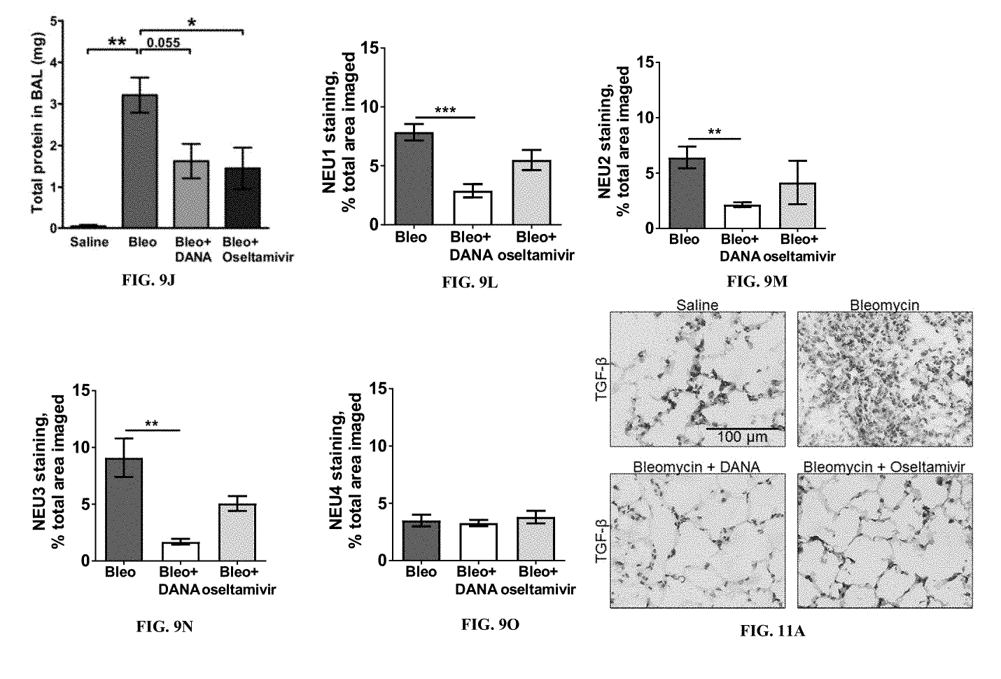

FIGS. 9A-9E show that daily intraperitoneal injections of the sialidase inhibitors decreased fibrosis in the mouse bleomycin model at day 21. FIG. 9A shows cryosections stained for collagen with 0.1% Sirius red (Polysciences, Warrington, Pa.) in saturated picric acid (Sigma) for 15 minutes. Images are representative of 3 mice per group. FIG. 9B shows quantification of the Sirius red staining. Compared to the saline control, bleomycin induced collagen deposition in the lungs (fibrosis), and this collagen deposition was reduced by treatment with either DANA or oseltamivir, indicating that inhibiting sialidase activity inhibits fibrosis.

FIG. 9C shows the total number of cells collected in BAL from the various mice. More cells were collected in BAL from the mice with bleomycin-induced fibrosis than those without fibrosis. Sialidase inhibitors reduced the number of cells collected. Since increased numbers of BAL cells indicates increased inflammation in the lung fluid, these results indicate that during fibrosis, sialidase inhibitors inhibit inflammation.

FIG. 9D shows the total number of CD11b+ cells and CD11c+ cells collected from the BAL. CD11b is a marker for inflammatory neutrophils and macrophages. CD11c is a marker for resident lung macrophages and dendritic cells. Mice with bleomycin-induced fibrosis had increased levels of cells with both markers. These levels were decreased by sialidase inhibitors, indicating that sialidase inhibitors were able to inhibit inflammation.

FIG. 9E shows the total protein in BAL from the various mice. Bleomycin resulted in an increase in total protein. This increased BAL fluid protein was attenuated by treatment with sialidase inhibitors, indicating that that the sialidase inhibitors inhibit edema and/or or epithelial barrier destruction during fibrosis.

Treatment of mice with oropharyngeal bleomycin results in an initial inflammation, and then a fibrosis that has begun by day 10 after bleomycin treatment. To determine if sialidase inhibitors decrease fibrosis when treatment starts after the initial inflammation stage and when fibrosis is present, mice were treated with oropharyngeal bleomycin to induce symptoms of pulmonary fibrosis as described above. Then, starting 10 days after bleomycin had been administered, the mice were given daily intraperitoneal injections of 10 mg/kg DANA (Calbiochem) in PBS or 10 mg/kg oseltamivir (Sigma) in PBS. At day 21, mice were sacrificed and BAL and cryosections of lungs were obtained as described above. The experiment was performed in accordance with the recommendations in the Guide for the Care and Use of Laboratory Animals of the National Institutes of Health. The Texas A&M University Animal Use and Care Committee approved the protocol.

FIG. 9F shows two sets of cryosections stained for collagen as in FIG. 9A. Images are representative of 3 mice per group. FIG. 9G shows quantification of the FIG. 9F staining. Compared to the saline control, bleomycin induced collagen deposition in the lungs (fibrosis), and this collagen deposition was reduced by treatment with either DANA or oseltamivir starting at day 10, indicating that inhibiting sialidase activity inhibits an existing fibrosis. FIG. 9H shows the total number of cells collected in BAL from the various mice. More cells were collected in BAL from the mice with bleomycin-induced fibrosis than those without fibrosis. Treatment with sialidase inhibitors starting at day 10 reduced the number of cells collected. Since increased numbers of BAL cells indicates increased inflammation in the lung fluid, these results indicate that when administered starting when a fibrosis has already been established, sialidase inhibitors inhibit inflammation.

FIG. 9I shows the total number of CD11b+ inflammatory neutrophils and macrophages collected from the BAL. Mice with bleomycin-induced fibrosis had increased levels of CD11b+ cells. These levels were decreased by treatment with sialidase inhibitors starting at day 10, indicating that sialidase inhibitors were able to inhibit inflammation when administered starting at a time after a fibrosis has become established.

FIG. 9J shows the total protein in BAL from the various mice. Bleomycin resulted in an increase in total protein. This increased BAL fluid protein was attenuated by treatment with the sialidase inhibitor oseltamivir beginning at day 10, indicating that a sialidase inhibitor inhibits edema and/or or epithelial barrier destruction during fibrosis when the administration of the sialidase inhibitor begins after a fibrosis has become established.

Overall, the data in FIGS. 9A-9J indicate that sialidase inhibitors may decrease fibrocyte formation and help prevent or inhibit fibrosis.

In mice treated as in FIGS. 9F-9J, decreased NEU1, NEU2 and NEU3 staining was also observed at 21 days. Results representative of three independent experiments are presented in FIG. 9K, with ImageJ quantification in FIGS. 9L-9O. The results indicate that sialidase inhibitors also decrease sialidase expression, consistent with inhibition of a positive feedback loop.

### Example 11

### Fibrotic Mouse Lungs Contain Normal Levels of Total Sialic Acid

To determine if the reduced levels of sialic acid on glycoconjugates in fibrotic mouse lungs was due to reduced levels of total sialic acid, the sialic acid content of pieces of lung tissue was determined. 0.2 g resorcinol was dissolved in 10 ml water. 1 ml of the 2% resorcinol stock solution was mixed with 8 ml of 12M HCl. 25 μl of 0.1 M CuSO₄ in water was added to this solution, and the volume was adjusted to 10 ml with water. Approximately 1.2×1.2×1.2 mm pieces from lungs frozen in OCT (and used for FIG. 8C) were collected. The OCT was allowed to thaw and the lung pieces were then washed by repeatedly pipetting 500 μl of PBS onto the sample; this was repeated with 3 aliquots of PBS. After removing the PBS, the lung piece was weighed. Lung pieces were placed in 200 ul of PBS in eppendorf tubes. Sialic acid (Vector laboratories) was weighed and dissolved in PBS to make a series of concentration standards. 200 μl of the resorcinol/HCl/CuSO₄ solution was added to the lung tissue pieces in PBS, and to 200 μl of standard solutions. Tubes were then incubated in a heating block at 100° C. for 15 minutes, and the tubes were then cooled to room temperature in a water bath. 0.5 ml of iso-amyl alcohol was added to the tubes and was mixed by vigorous shaking for 1 minute. The tubes were cooled in ice water for 15 minutes and then subjected to centrifugation for 2 minutes at 1000×g. 100 μl of the upper amyl alcohol phase was transferred to the well of a 96-well plate, and the absorbance was read at 450 nm and 580 nm. The absorbance at 580 nm was then subtracted from the absorbance at 450 nm, a standard curve was plotted, and the unknown concentration of sialic acid from the tissue samples were estimated from the curve. Values were then converted to mg sialic acid/g tissue. As shown in FIG. 10, there was no significant difference between the sialic acid content of the control (saline treated) and fibrotic (bleomycin-treated) lungs. This then supports the idea that the reduced levels of sialylation seen in fibrotic lungs is due to an increase level of sialidase activity.

### Example 12

### Sialidase Inhibitors Also Decrease TGF-β1 Levels in Mice

To determine if sialidase inhibitors could also reduce TGF-β1 levels in mice, mice were treated with bleomycin or saline, then injected daily with saline, DANA or oseltamivir starting at day 10 after bleomycin treatment, as described in Example 9. The mice were euthanized at day 21 and sections of lung tissue were stained for TGF-β1. Results are presented in FIG. 11A and are representative of results for three mice per group. FIG. 11B shows ImageJ quantification of the FIG. 11A staining.

Both DANA and oseltamivir reduced TGF-β1 levels in bleomycin-treated mice, consistent with the connection between TGF-β1 and sialidases.

The above disclosed subject matter is to be considered illustrative, and not restrictive, and the appended claims are intended to cover all such modifications, enhancements, and other embodiments which fall within the true spirit and scope of the present disclosure. For example, although the disclosure focuses on inhibiting human sialidases, the sialidase inhibitors disclosed may be effective against other mammalian sialidases, particularly those with a protein sequence or structure similar to human sialidase. Efficacy of sialidase inhibitors against other mammalian sialidases may be readily determined using the methods set forth in this disclosure. In addition, methods of affecting fibrocytes and fibrosis using such sialidase inhibitors may be adapted from this disclosure.

1. A method of preventing or inhibiting fibrosis in a human comprising administering to a human at least one of Compounds 1-58, or any combination thereof, in an amount and for a time sufficient to inhibit the activity of at least one human sialidase in the human.

2. The method of claim 1, wherein at least one of Compounds 1-58 or any combination thereof is administered.

3. The method of claim 1, wherein the activity of at least human NEU1 in desialylating SAP is inhibited.

4. The method of claim 1, wherein the activity of at least human NEU2 in desialylating SAP is inhibited.

5. The method of claim 1, wherein the activity of at least human NEU3 in desialylating SAP is inhibited.

6. The method of claim 1, wherein the activity of at least human NEU4 in desialylating SAP is inhibited.

7. The method of claim 1, wherein the activity of at least one human sialidase on terminal sialic acids with an α(2,6)-linkage is inhibited.

8. The method of claim 1, wherein the activity of at least one human sialidase on terminal sialic acids with an α(2,3)-linkage is inhibited.

9. The method of claim 1, wherein the formation or activation of fibrocytes is inhibited as a result of inhibition of human sialidase activity on SAP.

10. The method of claim 1, wherein at least one of Compounds 1-58 is administered to the human systemically.

11. The method of claim 1, wherein at least one of Compounds 1-58 is administered to the human locally in an area in which human sialidase activity is abnormally high.

12. The method of claim 1, wherein at least one of Compounds 1-58, or any combination thereof, is administered to the human in an amount and for a time sufficient to inhibit the activity of at least two human sialidases in the human.

13. A method of preventing or inhibiting fibrosis in a human comprising administering to a human at least one of Compounds 1-58, or any combination thereof, in an amount and for a time sufficient to inhibit level or activity of TGF-β1 in the human.

14. The method of claim 13, wherein at least one of Compounds 1-58 or any combination thereof is administered.

15. The method of claim 13, wherein at least one of Compounds 1-58, or any combination thereof, is administered to the human in an amount and for a time sufficient to additionally inhibit the activity of a human sialidase in the human.

16. The method of claim 15, wherein the activity of at least human NEU1 in desialylating SAP is inhibited.

17. The method of claim 15, wherein the activity of at least human NEU2 in desialylating SAP is inhibited.

18. The method of claim 15, wherein the activity of at least human NEU3 in desialylating SAP is inhibited.

19. The method of claim 15, wherein the activity of at least human NEU4 in desialylating SAP is inhibited.

20. The method of claim 15, wherein the activity of at least one human sialidase on terminal sialic acids with an α(2,6)-linkage is inhibited.

21. The method of claim 15, wherein the activity of at least one human sialidase on terminal sialic acids with an α(2,3)-linkage is inhibited.

22. The method of claim 13, wherein the proliferation or activation of fibroblasts is also inhibited as a result of inhibition of level or activity of TGF-β1.

23. The method of claim 1, wherein at least one of Compounds 1-58 is administered to the human systemically.

24. The method of claim 1, wherein at least one of Compounds 1-58 is administered to the human locally in an area in which human sialidase activity is abnormally high.

25. A method of preventing or inhibiting fibrosis in a human comprising administering to a human at least one isolated human or humanized monoclonal antibody that binds to the active site of at least one human sialidase wherein the antibody is administered in an amount and for a time sufficient to inhibit the activity of the at least one human sialidase in the human.

26. The method of claim 25, wherein the activity of at least human NEU1 in desialylating SAP is inhibited.

27. The method of claim 25, wherein the activity of at least human NEU2 in desialylating SAP is inhibited.

28. The method of claim 25, wherein the activity of at least human NEU3 in desialylating SAP is inhibited.

29. The method of claim 25, wherein the activity of at least human NEU4 in desialylating SAP is inhibited.

30. The method of claim 25, wherein the activity of at least one human sialidase on terminal sialic acids with an α(2,6)-linkage is inhibited.

31. The method of claim 25, wherein the activity of at least one human sialidase on terminal sialic acids with an α(2,3)-linkage is inhibited.

32. The method of claim 25, wherein the formation or activation of fibrocytes is inhibited as a result of inhibition of human sialidase activity on SAP.
